(19) United States
(12) Patent Application Publication
(10) Pub. No.: US 2024/0261215 A1
(43) Pub. Date: Aug. 8, 2024

(54) COSMETIC COMPOSITIONS CONTAINING GLYCEROLATED SILICONE RESIN AND HYDROGEL-IN-OIL DISPERSION

(71) Applicant: L'OREAL, Paris (FR)

(72) Inventors: Jody EBANKS, Clark, NJ (US); Komal LADD, Kenilworth, NJ (US); Tsang-Min HUANG, Scotch Plains, NJ (US); Susan HALPERN CHIRCH, Basking Ridge, NJ (US)

(21) Appl. No.: 18/162,378

(22) Filed: Jan. 31, 2023

(51) Int. Cl. A61K 8/892; A61K 8/31; A61K 8/81; A61Q 1/04

(52) U.S. Cl. CPC A61K 8/892; A61K 8/31; A61K 8/8147; A61Q 1/04; A61K 2800/43; A61K 2800/624; A61K 2800/654; A61K 2800/95

(57) ABSTRACT

A cosmetic composition including (i) hydrogel-in-oil dispersion, and (ii) at least one glycerolated silicone resin, as well as methods, kits and sets using or containing such compositions.

### TECHNICAL FIELD

The present invention relates to compositions, in particular lip compositions, comprising (1) at least one glycerolated silicone resin and (2) a hydrogel-in-oil dispersion, as well as to methods, kits and sets comprising such compositions.

### DISCUSSION OF THE BACKGROUND

Consumers expect that cosmetics used for extended periods of time, such as lipsticks, foundations and mascaras, should have excellent non-transfer properties against liquids such as olive oil, acetic acid and/or saliva, and should keep consistent wear (little or no smudging or smearing). To achieve these properties, such compositions are often devoid of skin or lip care ingredients (or contain very little) due to disruption of the film properties, which decrease the wear properties. It is a challenge to incorporate many skin or lip care ingredients and actives into such formulas. Further, controlling the release of such skin-care ingredients can be very difficult when they are incorporated into the products.

Such extended-wear products can also be expected to provide certain aesthetic properties. For example, lipsticks are expected to be comfortable to wear and provide a shine, matte, or satin finish to the lips as desired.

Finally, providing such features to an extended-wear product can be challenging if the product also requires providing volume, particularly through swelling.

The aim of the present invention is to propose compositions which offer excellent staying power of the expected cosmetic effects, notably the color of the makeup on keratin materials (skin, lips, nails, hair, eyelashes, eyebrows) which may extend the duration of the cosmetic composition on keratin materials, as well as improve wear of the cosmetic composition on keratin materials and its resistance to external forces such as mechanical friction, water, sweat and perspiration, sebum, oil, etc.

In addition, the aim of the present invention is to propose compositions which afford staying power of the expected cosmetic effects, notably the color of the makeup on keratin materials, combined with a good level of comfort in comparison with compositions containing conventional systems, in particular conventional systems based on unmodified silicone resin alone.

Accordingly, one aspect of the present invention is a composition, in particular a lip composition, which has improved properties with respect to composition ease of and/or comfort of application of the composition, and/or composition color stability particularly as it relates to improved wear (smudging and smearing) properties.

### SUMMARY OF THE INVENTION

The present invention relates to compositions, in particular lip compositions, comprising (1) at least one glycerolated silicone resin and (2) a hydrogel-in-oil dispersion. Preferably, the composition further comprises at least one active agent, in particular a water-soluble active agent, and/or at least one colorant. Preferably, the composition (1) is in the form of an emulsion, (2) is in liquid form, and/or (3) does not contain emulsion-stabilizing effective amounts of surfactants.

The present invention also relates to methods of caring for, and/or making up keratinous material by applying compositions of the present invention to the keratinous material in an amount sufficient to care for, and/or to make up the keratinous material.

The present invention also relates to kits comprising, as separate compositions in one or more containers within the kits, (A) a composition, in particular a lip composition, comprising (1) at least one glycerolated silicone resin and (2) a hydrogel-in-oil dispersion; and (B) at least one topcoat or primer coat composition. Preferably, the composition further comprises at least one active agent, in particular a water-soluble active agent, and/or at least one colorant. Preferably, the composition (1) is in the form of an emulsion, (2) is in liquid form, and/or (3) does not contain emulsion-stabilizing effective amounts of surfactants.

The present invention also relates to sets, as applied onto keratinous material, comprising (A) at least one first layer of at least one primer coat and/or topcoat; and (B) at least one second layer of at least one composition, in particular a lip composition, comprising (1) at least one glycerolated silicone resin and (2) a hydrogel-in-oil dispersion. Preferably, the composition further comprises at least one active agent, in particular a water-soluble active agent, and/or at least one colorant. Preferably, the composition (1) is in the form of an emulsion, (2) is in liquid form, and/or (3) does not contain emulsion-stabilizing effective amounts of surfactants. Preferably, the at least one second layer is applied over the at least one first layer (in which case the first layer is a primer coat composition). However, if the at least one second layer is applied under the at least one first layer, the at least one first layer is a topcoat layer. Preferably, the layer (B) further comprises at least one active agent, in particular a water-soluble active agent.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3,2-4, 1-4, etc.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Wax” as used herein is a lipophilic fatty compound that is solid at ambient temperature (25° C.) and changes from the solid to the liquid state reversibly, having a melting temperature of more than 30° C. and, for example, more than 45ºC, and a hardness of more than 0.5 MPa at ambient temperature.

“Surfactant” and “emulsifier” are used interchangeably throughout this specification.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of wax” means that an effective amount (that is, more than trace amounts) of wax is omitted from the composition (that is, about 0% by weight), “substantially free of wax” means that wax is are present in amounts not greater than 0.1% by weight, and “devoid of wax” means that wax is present in amounts not greater than 0.25% by weight, based on the total weight of the composition. The same nomenclature applies for all other ingredients identified throughout the application and in this paragraph such as, for example, water (compositions of the invention which are “free of water,” “substantially free of water,” and “devoid of water” have meanings consistent with the discussion within this paragraph), and surfactant (compositions of the invention which are “free of surfactant,” “substantially free of surfactant,” and “devoid of surfactant” have meanings consistent with the discussion within this paragraph), even if not specifically discussed for each identified ingredient. Discussed examples of the use of such language are intended to be exemplary, not limiting.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as lips and evaluating the color of the composition after an extended period of time. For example, the color of a composition may be evaluated immediately following application to keratin materials such as lips and these characteristics may then be re-evaluated and compared after a certain amount of time. Further, these characteristics may be evaluated with respect to other compositions, such as commercially available compositions.

“Keratinous material” or “keratin material” means natural nails, lips, skin such as the face, the body, the hands, and the area around the eyes, and keratin fibres such as head hair, eyelashes, eyebrows, bodily hair of a human, as well as synthetic additions such as false eyelashes, false eyebrows, false nails, etc.

“Physiologically acceptable” means compatible with keratinous material and having a pleasant color, odor and feel, and which does not cause any unacceptable discomfort (stinging or tautness) liable to discourage a consumer from using the composition.

“Room temperature” means 25° C.“Atmospheric pressure” means 760 mmHg, i.e. 105 pascals.

As used herein, the term “alkyl radical” refers to a linear or branched saturated C1-C8, in particular C₁-C₆, preferably C1-C4 hydrocarbon-based group such as methyl, ethyl, n-propyl, isopropyl, n-butyl, isobutyl and tert-butyl.

As used herein, the term “(C9-C22)alkyl” radical refers to a linear or branched, preferably linear, saturated C9-C22, in particular C10-C20, preferentially C12-C18, more preferentially C12-C16 hydrocarbon-based group, such as lauryl (C12), myristyl (C14), hexadecyl (C16), stearyl (C18), arachidyl (C20) or behenyl (C22); more particularly, (C9-C18)alkyl is a linear or branched, preferably linear, saturated C9-C18 hydrocarbon-based group.

As used herein, the term “alkylene” radical refers to a linear or branched divalent saturated C1-C8, in particular C₁-C₆, preferably C₁-C₄ hydrocarbon-based group such as methylene, ethylene or propylene.

As used herein, the term “(di) (C₁-C₄) (alkyl) amino” refers to an amino radical —NH2; a (C₁-C₄) alkylamino radical such as methylamino ethylamino; a di (C₁-C₄) alkylamino radical such as dimethylamino, diethylamino, preferably dimethylamino.

As used herein, the term “anhydrous” dispersion or composition means a dispersion or composition containing less than 2% by weight of water, or even less than 0.5% of water, and notably free of water; where appropriate, such small amounts of water may notably be provided by ingredients of the composition which may contain residual amounts thereof.

As used herein, the term “aryl” means a monocyclic or fused or non-fused polycyclic carbon-based group comprising from 6 to 22 carbon atoms, and in which at least one ring is aromatic. In some embodiments, the aryl radical refers to a monocyclic or fused or non-fused bicyclic, unsaturated cyclic aromatic radical comprising from 6 to 12 carbon atoms; preferably, the aryl group comprises 1 ring and contains 6 carbon atoms, such as phenyl. In some embodiments, the aryl radical is a phenyl, biphenyl, naphthyl, indenyl, anthracenyl or tetrahydronaphthyl.

As used herein, the term “aryloxy” radical refers to an aryl-oxy, i.e. aryl-O—, radical, with aryl as defined previously, preferably phenoxy.

As used herein, the term “aryl(C₁-C₄)alkoxy” radical refers to an aryl-(C₁-C₄)alkyl-O-radical, preferably benzoxy.

As used herein, the term “cyclic” radical refers to a cyclic saturated or unsaturated, aromatic or non-aromatic hydrocarbon-based group comprising from 1 to 3 rings, preferably 1 ring, and comprising from 3 to 10 carbon atoms, such as cyclohexyl or phenyl.

As used herein, the term “cycloalkyl” radical refers to a cyclic saturated hydrocarbon-based group comprising from 1 to 3 rings, preferably 2 rings, and comprising from 3 to 12 carbon atoms, preferably between 5 and 10 carbon atoms, such as cyclopentyl, cyclohexyl, cycloheptyl, norbornyl, or isobornyl, the cycloalkyl radical possibly being substituted with one or more (C₁-C₄)alkyl groups such as methyl; preferably, the cycloalkyl radical is an isobornyl group.

As used herein, the term “insoluble” monomer refers to any monomer of which the polymer, notably the homopolymer, is not in soluble form, i.e. not fully dissolved at a concentration of greater than 5% by weight at room temperature (20° C.) in said liquid hydrocarbon-based medium consisting of iii) liquid hydrocarbon-based fatty substances. However, the “insoluble” monomers may, as monomers, be soluble in the liquid hydrocarbon-based medium consisting of liquid hydrocarbon-based fatty substances iii) of the dispersion, it being understood that they become insoluble after polymerization.

As used herein, the term “soluble” monomer refers to any monomer of which the polymer, notably the homopolymer, is soluble, to 5% by weight, at 20° C. and at atmospheric pressure in the liquid hydrocarbon-based medium consisting of liquid hydrocarbon-based fatty substances iii) of the dispersion. The polymer, notably the homopolymer, is completely dissolved in the liquid carbon-based medium, visually at 20° C., i.e., no insoluble deposit or precipitate or agglomerate or sediment is visually noted.

As used herein, the term “ethylenic copolymer” means a polymer derived from the polymerization of different monomers, in particular at least two different monomers. Preferably, the ethylenic copolymer of the invention is derived from two or three different monomers, more .preferentially derived from two different monomers.

As used herein, the term “ethylenic homopolymer” means a polymer derived from the polymerization of identical monomers.

As used herein, the term “ethylenic monomer” means an organic compound including one or more conjugated or non-conjugated unsaturations of >C=C<type, which is capable of polymerizing.

As used herein, the term “fatty substance” means an organic compound that is immiscible in water at ordinary room temperature (25° C.) and at atmospheric pressure (760 mmHg) (solubility of less than 5%, preferably 1% and even more preferentially 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance ethanol, ether, liquid petroleum jelly or decamethylcyclopentasiloxane. These fatty substances are neither polyoxyethylenated nor polyglycerolated. They are different from fatty acids, since salified fatty acids constitute soaps that are generally soluble in aqueous media.

As used herein, the term “liquid” fatty substance notably refers to a fatty substance that is liquid at 25° C. and 1 atmosphere; preferably, said fatty substance has a viscosity of less than or equal to 7000 centipoises at 20° C.

As used herein, the term “hydrocarbon-based” fatty substance means a fatty substance which comprises at least 50% by weight, notably from 50% to 100% by weight, for example from 60% to 99% by weight, or even from 65% to 95% by weight, or even from 70% to 90% by weight, relative to the total weight of said fatty substance, of carbon-based compound, having a global solubility parameter in the Hansen solubility space of less than or equal to 20 (MPa)½, or a mixture of such compounds.

As used herein, the global solubility parameter δ according to the Hansen solubility space is defined in the article “Solubility parameter values” by Grulke in the book “Polymer Handbook”, 3rd Edition, Chapter VII, pages 519-559, by the relationship δ=(dD2+dP2+dH2)½ in which: -dD characterizes the London dispersion forces arising from the formation of dipoles induced during molecular impacts, -dP characterizes the Debye interaction forces between permanent dipoles, -dH H characterizes the forces of specific interactions (such as hydrogen bonding, acid/base, donor/acceptor, etc.); The definition of solvents in the Hansen three-dimensional solubility space is described in the article by Hansen: The three-dimensional solubility parameters, J. Paint Technol. 39, 105 (1967).

As used herein, the term “oil” means a fatty substance that is liquid at room temperature (25° C.) and at atmospheric pressure.

As used herein, the term “hydrocarbon-based oil” means an oil formed essentially from, or even constituted of, carbon and hydrogen atoms, and optionally of oxygen and nitrogen atoms, and not containing any silicon or fluorine atoms. It may contain hydroxy, ester, ether, carboxylic acid, amine and/or amide groups.

As used herein, the term “volatile oil” means an oil (or non-aqueous medium) that can evaporate on contact with keratin materials, in particular the skin, in less than one hour, at room temperature and at atmospheric pressure. The volatile oil is a volatile cosmetic oil, which is liquid at room temperature, notably having a non-zero vapour pressure, at room temperature and at atmospheric pressure, in particular having a vapour pressure ranging from 0.13 Pa to 40 000 Pa (10-3 to 300 mmHg), preferably ranging from 1.3 Pa to 13 000 Pa (0.01 to 100 mmHg) and preferentially ranging from 1.3 Pa to 1300 Pa (0.01 to 10 mmHg).

As used herein, the term “non-volatile oil” means an oil with a vapour pressure of less than 0.13 Pa at room temperature and at atmospheric pressure.

As used herein, the term “silicone oil” means an oil comprising at least one silicon atom and notably at least one Si—O group. The silicone oil may be volatile or non-volatile.

As used herein, the term “dispersant” refers to a compound which can protect the dispersed particles from agglomerating or flocculating. This dispersant may be a surfactant, an oligomer, a polymer or a mixture of several thereof, bearing one or more functionalities with strong affinity for the surface of the ethylenic polymeric core particles to be dispersed; in particular, they can attach physically or chemically to the surface of the pigments. These dispersants also contain at least one functional group that is compatible with or soluble in the continuous medium. Said agent may be charged: it may be anionic, cationic, zwitterionic or neutral.

As used herein, the term “submicron” or “submicronic” refers to pigments having a particle size that has been micronized by a micronization method and having a mean particle size of less than a micrometer (μm), in particular between 0.1 and 0.9 μm, and preferably between 0.2 and 0.6 μm.

As used herein, the term “heteroaryl” means an optionally cationic, 5- to 22-membered, monocyclic or fused or non-fused polycyclic group, comprising from 1 to 6 heteroatoms chosen from nitrogen, oxygen, sulfur and selenium, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthyl and the ammonium salt thereof.

As used herein, the term “heterocyclic” means a 5- to 22-membered, monocyclic or fused or non-fused polycyclic aromatic or non-aromatic radical that may contain one or more unsaturations, including from 1 to 6 heteroatoms chosen from nitrogen, oxygen and sulfur.

As used herein, the term “heterocycloalkyl” means a saturated heterocyclic radical such as morpholinyl, piperazinyl or piperidyl.

As used herein, the term “organic or mineral acid salt” more particularly means salts chosen from a salt derived from a halogenated acid such as i) hydrochloric acid HCl, ii) hydrobromic acid HBr, iii) sulfuric acid H2SO4, iv) alkylsulfonic acids: Alk-S(O)2OH such as methylsulfonic acid and ethylsulfonic acid; v) arylsulfonic acids: Ar—S(O)2OH such as benzenesulfonic acid and toluenesulfonic acid; optionally hydroxylated carboxylic acids such as vi) citric acid; vii) succinic acid; viii) tartaric acid; ix) lactic acid; x) acetic acid CH3C(O)OH; xi) alkoxysulfinic acids: Alk-O—S(O)OH such as methoxysulfinic acid and ethoxysulfinic acid; xii) aryloxysulfinic acids such as tolueneoxysulfinic acid and phenoxysulfinic acid; xiii) phosphoric acid H3PO4; xiv) triflic acid CF3SO3H and xv) tetrafluoroboric acid HBF4; more preferentially, the organic or mineral acid salts are chosen from the salts of halogenated acids such as HCl and HBr, and of optionally hydroxylated carboxylic acids such as vi) citric acid; vii) succinic acid; viii) tartaric acid; ix) lactic acid; x) acetic acid CH3C(O)OH.

As used herein, the term “anionic counterion” means an anion or an anionic group derived from an organic or mineral acid salt which counterbalances the charge of the molecule in question; more particularly, the anionic counterion is chosen from: i) halides such as chloride or bromide; ii) nitrates; iii) sulfonates, including C1-C6 alkylsulfonates: Alk-S(O)20—such as methylsulfonate or mesylate and ethylsulfonate; iv) arylsulfonates: Ar—S(O)20—such as benzenesulfonate and toluenesulfonate or tosylate; v) citrate; vi) succinate; vii) tartrate; viii) lactate; ix) alkyl sulfates: Alk-O—S(O)O— such as methyl sulfate and ethyl sulfate; x) aryl sulfates: Alk-O—S(O)O— such as benzene sulfate and toluene sulfate; xi) alkoxy sulfates: Alk-O—S(O)20-such as methoxy sulfate and ethoxy sulfate; xii) aryloxy sulfates: Ar—O—S(O)2O—, xiii) phosphates O=P(OH)2—O—, O=P(O—)2-OH, O=P(O—)3, HO-[P(O)(O—)]w-P(O)(O—)2 with w being an integer; xiv) acetate; xv) triflate; and xvi) borates such as tetrafluoroborate, xvii) disulfate (O═)2S(O—)2 or SO42—and monosulfate HSO4-; the anionic counterion, derived from an organic or mineral acid salt, ensures the electrical neutrality of the molecule; thus, it is understood that when the anion comprises several anionic charges, then the same anion can serve for the electrical neutrality of several cationic groups in the same molecule or else may serve for the electrical neutrality of several molecules; for example, a molecule which contains two cationic groups may contain either two “singly charged” anionic counterions or contain a “doubly charged” anionic counterion such as (O═)2S(O—)2 or O=P(O—)2-OH.

Moreover, the addition salts that may be used in the context of the present disclosure are notably chosen from addition salts with a cosmetically acceptable base such as the basifying agents as defined below, for instance alkali metal hydroxides such as sodium hydroxide, potassium hydroxide, aqueous ammonia, amines or alkanolamines.

For the purposes of the present disclosure, and unless otherwise indicated:
the “aryl” or “heteroaryl” radical or the aryl or heteroaryl part of a radical may be substituted with at least one substituent borne by a carbon atom, chosen from:
a C₁-C₆ and preferably C₁-C₄ alkyl radical optionally substituted with one or more radicals chosen from hydroxyl, C₁-C₂ alkoxy, C₂-C₄ (poly)hydroxyalkoxy, acylamino, amino substituted with two C₁-C₄ alkyl radicals, which may be identical or different, optionally bearing at least one hydroxyl group, or the two radicals possibly forming, with the nitrogen atom to which they are attached, a saturated or unsaturated, optionally substituted 5- to 7 membered and preferably 5- or 6-membered heterocycle optionally comprising another nitrogen or non-nitrogen heteroatom;
halogen;
hydroxyl;
C1-C2 alkoxy;
C2-C4 (poly)hydroxyalkoxy;
amino;
an amino radical substituted with one or two identical or different C₁-C₆ alkyl radicals, optionally bearing at least one hydroxyl group;
acylamino (—NR—C(O)—R′) in which the radical R is a hydrogen atom or a C₁-C₄ alkyl radical optionally bearing at least one hydroxyl group and the radical R′ is a C1-C2 alkyl radical;
carbamoyl ((R)2N—C(O)—) in which the radicals R, which may be identical or different, represent a hydrogen atom or a C₁-C₄ alkyl radical optionally bearing at least one hydroxyl group;
alkylsulfonylamino (R′—S(O)₂—N(R)—) in which the radical R represents a hydrogen atom or a C₁-C₄ alkyl radical optionally bearing at least one hydroxyl group and the radical R′ represents a C₁-C₄ alkyl radical, or a phenyl radical;
an aminosulfonyl radical ((R)2N—S(O)2-) in which the radicals R, which may be identical or different, represent a hydrogen atom or a C₁-C₄ alkyl radical optionally bearing at least one hydroxyl group;
carboxylic in acid or salified (preferably with an alkali metal or a substituted or
unsubstituted ammonium) form;
cyano;
nitro or nitroso;
polyhaloalkyl, preferentially trifluoromethyl;
the cyclic or heterocyclic part of a non-aromatic radical may be substituted with at least one substituent chosen from the following groups: a) hydroxyl; b) C₁-C₄ alkoxy, C2-C4 (poly)hydroxyalkoxy; c) C₁-C₄ alkyl;
alkylcarbonylamino (R—C(O)—N(R′)-) in which the radical R′ is a hydrogen atom or a C₁-C₄ alkyl radical optionally bearing at least one hydroxyl group, and the radical R is a C1-C2 alkyl radical or an amino radical optionally substituted with one or two C₁-C₄ alkyl groups, which may be identical or different, themselves optionally bearing at least one hydroxyl group, said alkyl radicals possibly forming, with the nitrogen atom to which they are attached, a saturated or unsaturated, optionally substituted 5- to 7-membered heterocycle optionally comprising at least one other nitrogen or non-nitrogen heteroatom;
alkylcarbonyloxy (R—C(O)—O—) in which the radical R is a C₁-C₄ alkyl radical or an amino group optionally substituted with one or two identical or different C₁-C₄ alkyl groups themselves optionally bearing at least one hydroxyl group, said alkyl radicals possibly forming with the nitrogen atom to which they are attached a saturated or unsaturated, optionally substituted 5- to 7-membered heterocycle, optionally comprising at least one other nitrogen or non-nitrogen heteroatom;
alkoxycarbonyl (R-G-C(O)—) in which the radical R is a C₁-C₄ alkoxy radical, G is an oxygen atom or an amino group optionally substituted with a C₁-C₄ alkyl group itself optionally bearing at least one hydroxyl group, said alkyl radical possibly forming with the nitrogen atom to which they are attached a saturated or unsaturated, optionally substituted 5- to 7-membered heterocycle, optionally comprising at least one other nitrogen or non-nitrogen heteroatom;
a cyclic or heterocyclic radical, or a non-aromatic part of an aryl or heteroaryl radical, may also be substituted with one or more oxo groups; and
a hydrocarbon-based chain is unsaturated when it includes one or more double bonds and/or one or more triple bonds.

The compositions and methods of the present invention can comprise, consist of, or consist essentially of the essential elements and limitations of the invention described herein, as well as any additional or optional ingredients, components, or limitations described herein or otherwise useful. For example, the film forming component can “consist essentially of” the at least one glycerolated silicone resin and the hydrogel-in-oil dispersion, and/or the oil component can “consist essentially of” volatile hydrocarbon oil.

For purposes of the present invention, the “basic and novel property” associated with compositions, components and methods which “consist essentially of” identified ingredients or actions is “transfer-resistance against saliva and oil.”

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Hydrogel-In-Oil Dispersion

In accordance with the present invention, compositions comprising a hydrogel-in-oil dispersion are provided. There are three components of the dispersion: (a) an ethylenic polymeric core particle, (b) a fatty substance that is liquid at 20° C. and 1 atmosphere, and (c) a polymeric stabilizing agent (“stabilizers”). Such dispersions are described in French patent application no. FR 2113186 and U.S. provisional patent application No. 62/287,650, both filed on Dec. 9, 2021, both of which are incorporated by reference herein in their entirety.

In some embodiments, the dispersion may be present from at least about 5 wt. % based on the total weight of the cosmetic composition.

The dispersion, combined with a wide range of film formers, can surprisingly improve transfer resistance while maintaining wear resistance. Further, the dispersion is a Pickering dispersion and the core particles form a hydrogel. By incorporating hydrophilic materials into the hydrogel, the hydrogel swells, which provides volume.

### Polymer Particles

To obtain the dispersion, one can polymerize particular monomers that are capable of forming the polymer “core” in the presence of a polymeric statistical stabilizer comprising in minor amount a part that is soluble and in major amount a part that is insoluble in the dispersion medium, i.e., in the liquid hydrocarbon-based fatty substance(s).

In some embodiments, the cosmetic composition includes a plurality of ethylenic polymeric core particles. In some embodiments, each ethylenic polymeric core particle has a single composition. In some embodiments, the plurality of ethylenic polymeric core particles includes at least two particles with different compositions.

In some embodiments, the ethylenic polymeric core particles of the dispersion may be made of ethylenic monomers of:
a₁) (C₁-C₄)alkyl (C₁-C₄)(alkyl)acrylate in which the (C₁-C₄) alkyl group(s) are optionally substituted by one or more group (s) chosen from hydroxy, and (di) (C₁-C₄) (alkyl) amino; and/or
a₂) poly[oxy(C₁-C₄)alkylene] (C₁-C₄)(alkyl)acrylate, and
a₃) ethylenic monomers comprising one or more carboxy, anhydride, phosphoric acid, sulfonic acid groups.

According to another particular embodiment of the instant disclosure, the ethylenic polymeric core particle of the dispersion may be made up of several polymer (s) chosen from among
b₁) at least one copolymer of ethylenic monomers of:
a₁) (C₁-C₄)alkyl (C₁-C₄)(alkyl)acrylate in which the (C₁-C₄) alkyl group (s) are optionally substituted by one or more group (s) chosen from hydroxy, and (di) (C₁-C₄) (alkyl) amino; and/or
a₂) poly[oxy(C₁-C₄)alkylene] (C₁-C₄)(alkyl)acrylate, and
a₃) ethylenic monomers comprising one or more carboxy, anhydride, phosphoric acid, sulfonic acid groups; and
b₂) at least one polymer of ethylenic monomers comprising one or more carboxy, anhydride, phosphoric acid or sulfonic acid groups.

Preferably, the ethylenic monomers comprising one or more carboxy, anhydride, phosphoric acid, sulfonic acid a3) groups are chosen from ethylenic monomers comprising one or more carboxy groups, more preferably (C₁-C₄) (alkyl) acrylic acids such as (meth) acrylic acid, in particular acrylic acid. Preferably, the ethylenic monomers of (C₁-C₄)alkyl (C₁-C₄)(alkyl)acrylate in which (C₁-C₄) alkyl denote (meth) acrylate of (C₁-C₄) alkyl such as ethyl (meth) acrylate or (methyl meth) acrylate, in particular methyl acrylate and ethyl acrylate.

Preferably, the ethylenic monomers of (C₁-C₄)alkyl (C₁-C₄)(alkyl)acrylate in which the (C₁-C₄) alkyl group or groups are substituted by one or more groups chosen from hydroxy, (di) ((C₁-C₄) alkyl) amino are chosen from ethylenic monomers of (C₁-C₄) (C₁-C₄) alkyl (alkyl) acrylate substituted with a hydroxy group or with a (di) ((C₁-C₄) group) alkyl) amino. According to one embodiment, the ethylenic monomers of (C₁-C₄) (C₁-C₄) alkyl (alkyl) acrylate in which the (C₁-C₄) alkyl group (s) are substituted by one or more hydroxy groups, are substituted by a hydroxy group such as 2-hydroxyethyl (meth) acrylate, in particular 2-hydroxyethyl acrylate (HEA).

According to another embodiment, the ethylenic monomers of (C₁-C₄)alkyl (C₁-C₄)(alkyl)acrylate in which the (C₁-C₄) alkyl group (s) are optionally substituted by one or more group (s) chosen from hydroxy, and (di) (C1-C4) (alkyl) amino, are substituted by a di (C₁-C₄) alkyl) amino group such as a dimethylamino group such as 3-(dimethylamino) propyl (meth) acrylate, 2-(dimethylamino) ethyl (meth) acrylate.

According to a particular embodiment of the instant disclosure, the ethylenic polymeric core particles of the dispersion contain particles A1 consisting of copolymers of ethylenic monomers:
a₁) (C₁-C₄)alkyl (C₁-C₄)(alkyl)acrylate in which the (C₁-C₄) alkyl group (s) are optionally substituted by one or more group (s) chosen from hydroxy, and (di) (C1-C4) (alkyl) amino; and/or
a₂) poly[oxy(C₁-C₄)alkylene] (C₁-C₄)(alkyl)acrylate, and
a₃) ethylenic monomers comprising one or more carboxy, anhydride, phosphoric acid, sulfonic acid,
preferably consisting of copolymers of ethylenic monomers a1) and a3), or a2) and a3), more preferably a1) and a3).

According to a particular embodiment of the instant disclosure, the ethylenic polymeric core particles of the dispersion may contain particles A′l consisting of copolymers of ethylenic monomers a′1) (C₁-C₄)alkyl (C₁-C₄)(alkyl)acrylate and a3) ethylenic monomers comprising one or more carboxy, anhydride, phosphoric acid, sulfonic acid.

According to another particular embodiment of the instant disclosure, the ethylenic polymeric core particles of the dispersion may contain particles A′2 consisting of copolymers of ethylenic monomers a″1) (C₁-C₄) (alkyl) (C₁-C₄) alkyl acrylate substituted with one or more groups chosen from hydroxy, (di) ((C₁-C₄) alkyl) amino, preferably substituted with a hydroxy group such as 2-hydroxyethylacrylate (HEA) and a3) monomers ethylenic comprising one or more carboxy, anhydride, phosphoric acid, sulfonic acid.

According to a preferred embodiment, the ethylenic polymeric core particles of the dispersion may contain particles A′1 and particles A′2 as described above and more preferably the ethylenic polymeric core particles of the dispersion are a mixture of particles A′l and A′2, in particular in a mass ratio (mass of particles A′1/mass of particles A′2) of between 0.3 and 3, more particularly between 0.5 and 2.8, even more preferably between 0.6 and 2.

According to another particular embodiment of the instant disclosure the ethylenic polymeric core particles of the dispersion may be made up of a mixture of b1) at least one copolymer of ethylenic monomers of a1) and/or a2) and a3) as described above and b2) at least one polymer of ethylenic monomers comprising one or more carboxy, anhydride, phosphoric acid or sulfonic acid groups.

According to another particular embodiment of the instant disclosure the ethylenic polymeric core particles of the dispersion may be made up of a mixture of b1) at least one copolymer of ethylenic monomers of a1) and a3) as described above and b2) at least one polymer of ethylenic monomers comprising one or more carboxy, anhydride, phosphoric acid or sulfonic acid groups.

According to a particular form of the instant disclosure, b2) denotes a homopolymer of ethylenic monomers comprising one or more carboxy, or anhydride, or phosphoric acid or sulfonic acid groups, preferably comprising a carboxy group, more preferably acid (meth) acrylic and even more preferably acrylic acid.

The ethylenic polymeric core particles can be crosslinked or uncrosslinked.

According to one embodiment of the instant disclosure, the ethylenic polymeric core particles contain ethylenic copolymers A′1 resulting from the polymerization of monomer of formula (I) with ethylenic monomers comprising one or more carboxy, anhydride, phosphoric acid, sulfonic acid a3 groups.

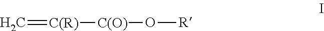

in which formula (I): R represents a hydrogen atom or a (C₁-C₄)alkyl group such as methyl, and R′ represents a (C₁-C₄)alkyl group such as methyl or ethyl, preferably a C₁-C₄ alkyl acrylate such as methyl acrylate.

According to a particular embodiment of the instant disclosure the (C₁-C₄) (C1-C4) (alkyl) acrylate monomers denote the C₁-C₄ alkyl (meth) acrylate monomers preferably chosen from methyl (meth) acrylate, ethyl (meth) acrylate, n-propyl (meth) acrylate, isopropyl (meth) acrylate, n-butyl (meth) acrylate, (meth) acrylate isobutyl, tert-butyl (meth) acrylate and more preferably chosen from methyl (meth) acrylate, ethyl (meth) acrylate and even more preferably chosen from methyl acrylate and ethyl acrylate.

According to an advantageous embodiment of the instant disclosure, the dispersion may include from 60% to 99% by weight, in particular from 70% to 99% by weight, preferably from 80% to 95% by weight, in particular from 85% to 93% by weight of monomers constituting the ethylenic polymeric core particles relative to the total weight of polymers contained in said dispersion.

Preferably, the monomers capable of forming the ethylenic polymeric core particles are chosen from monomers which are insoluble in the hydrocarbon-based liquid fatty substance(s) of the dispersion.

In some embodiments, the insoluble monomers may preferably represent from 80 to 100% by weight, more preferably from 90 to 100% by weight, even more preferably from 95% to 100% by weight and even more particularly 100% by weight, of the total weight of monomers forming the ethylenic polymeric core particles.

Ethylenic monomers bearing an acid, anhydride or aryl function:

The ethylenic polymeric core particles may comprise ethylenic polymers that comprise or consist of ethylenic monomers a3) comprising one or more carboxy, anhydride, phosphoric acid or sulfonic acid groups.

More particularly the ethylenic monomers a3) may comprise one or more carboxy, anhydride, phosphoric acid or sulfonic acid groups are chosen from (1), (2), (3) and (4):
(1) R¹(R²)C=C(R³)-Acid with R¹, R² and R³ representing a hydrogen atom or a CO₂H, H₂PO₄ or SO₃H group, and Acid representing a carboxyl, phosphoric acid or sulfonic acid group, preferably carboxyl, and also the organic or mineral base salts thereof such as the alkali metal or alkaline-earth metal salts, such as C(O)ONa or C(O)OK, preferably (1) represents (5) H2C=C(R)—C(O)—O—H with R representing a hydrogen atom or a (C₁-C₄)alkyl group such as methyl, and also the organic or mineral base salts thereof such as the alkali metal or alkaline-earth metal salts, such as Na or K;
(2) H₂C=C(R)—C(O)—N(R′)-Alk-Acid with R and R′, which may be identical or different, representing a hydrogen atom or a (C₁-C₄)alkyl group; Alk represents a (C₁-C₆)alkylene group optionally substituted with at least one group chosen from Acid as defined previously and hydroxyl; and Acid is as defined previously, preferably carboxyl or sulfonic acid, and also the organic or mineral base salts thereof such as the alkali metal or alkaline-earth metal salts such as C(O)ONa or C(O)OK;
(3) Ar—(R^{a})C=C(R^{b})—R^{c} with R^{a}, R^{b} and R^{c}, which may be identical or different, representing a hydrogen atom or a (C₁-C₄)alkyl group, and Ar representing an aryl group, preferably benzyl, optionally substituted with at least one acid group CO₂H, H₂PO₄ or SO₃H, preferably substituted with a CO₂H or SO₃H group, and also the organic or mineral base salts thereof such as the alkali metal or alkaline-earth metal salts such as C(O)ONa or C(O)OK;
(4) maleic anhydride of formulae (4a) and (4b):

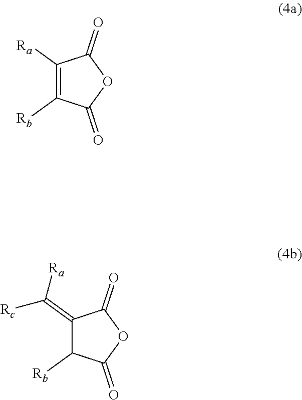

in which formulae (4b) and (4b) Rₐ, R_{b} and R_{c}, which may be identical or different, represent a hydrogen atom or a (C₁-C₄)alkyl group; preferably, Rₐ, R_{b} and R_{c} represent a hydrogen atom. Preferentially, the ethylenically unsaturated anhydride monomer is of formula (4b) and more preferentially is maleic anhydride.

More particularly, in some embodiments, the ethylenic monomer a3) comprising one or more carboxy, anhydride, phosphoric acid or sulfonic acid groups is chosen from (1) and (4), in particular (5) and more particularly (5) and even more preferably acrylic acid.

According to another particular embodiment of the instant disclosure, the polymer constituting the ethylenic polymeric core particles and notably A′1 and/or b1) is an ethylenic acrylate copolymer derived from the polymerization:
at least one monomer a1) of formula (I) as defined previously, preferably a C₁-C₄ alkyl acrylate such as methyl acrylate or ethyl acrylate; and
at least one monomer a3) of formula (5) and also the organic or mineral base salts thereof such as the alkali metal or alkaline-earth metal salts such as Na or K:

In some embodiments, the amount of acrylic acid ranges from 0.01% to 30% by weight relative to the total weight of copolymer A′1 or b1, preferably between 0.1% and 20% by weight relative to the weight of the polymer(s) of the ethylenic core particles. More particularly A′1 or b1), is in particular a copolymer resulting from the copolymerization of acrylic acid with one or more C₁-C₄ alkyl (meth) acrylate monomers, in particular chosen from methyl (meth) acrylate, and ethyl (meth) acrylate.

According to another embodiment, the polymer constituting the ethylenic polymeric core particles and notably A′1) and/or b1) is an ethylenic acrylate copolymer derived from the polymerization:
of at least two different monomers a1): of formula (I) as defined previously, preferably a C₁-C₄ alkyl (meth)acrylate such as methyl (meth)acrylate or ethyl (meth)acrylate; and
of a monomer a3) of formula (5) as defined previously, as well as its salts of organic or inorganic bases such as alkalis or alkaline-earth metals such as Na or K, preferably acrylic acid. Even more preferably A′1 and/or b1) results from the polymerization of methyl acrylate, ethyl acrylate and acrylic acid. According to one embodiment, the ethylenic monomer a3) comprising one or more carboxy, anhydride, phosphoric acid, sulfonic acid groups is chosen from crotonic acid, maleic anhydride, itaconic acid, fumaric acid, maleic acid, styrenesulfonic acid, vinylbenzoic acid, vinylphosphoric acid, acrylic acid, methacrylic acid, acrylamidopropanesulfonic acid, acrylamidoglycolic acid, and their salts, even more preferably the monomer ethylenic comprising one or more carboxy, anhydride, phosphoric acid, sulfonic acid represents acrylic acid and its salts.

According to one embodiment, the dispersion includes at least one type of particle. In some embodiments, the dispersion includes at least two types of different particles. In some embodiments, the dispersion includes 2 to 5 types of particles. In some embodiments, the dispersion includes two types of different particles.

In some embodiments, the dispersion comprises at least 2 types of different core particles, preferably two types of core particles which are copolymers of different chemical structure derived from monomers a₁) and/or a₂) and a₃), in particular which are copolymers of different chemical structure obtained by polymerization of monomers a₁) and a₃).

In some embodiments, the different core particles preferably comprise ethylenic copolymers (IA) of:
a″1) (C₁-C₄)alkyl (C₁-C₄)(alkyl)acrylate in which the (C₁-C₄) alkyl group (s) are optionally substituted by one or more group (s) chosen from hydroxy, and (di) (C₁-C₄) (alkyl) amino, preferably hydroxy and/or
a₂) oly[oxy(C₁-C₄)alkylene] (C₁-C₄)(alkyl)acrylate, and
a₃) ethylenic monomers comprising one or more carboxy, anhydride, phosphoric acid, sulfonic acid, preferably carboxy groups; preferably ethylenic copolymers of a′₁) and a₃).

More particularly the ethylenic monomers a3) comprising one or more carboxy, anhydride, phosphoric acid, sulfonic acid groups are chosen from (1), (2), (3), and (4) as defined above and denote in particular (5) as well as its salts of organic or inorganic bases such as alkalis or alkaline earth metals such as Na or K.

In some embodiments, the different core particles preferably comprise i) ethylenic copolymers (I′A) resulting from the polymerization of:
a′″₁ of at least one monomer of formula (I′) H₂C=C(R)—C(O)—O—R″, in which R represents a hydrogen atom or a (C₁-C₄)alkyl group such as methyl, and R″ represents a (C₁-C₄) alkyl group substituted with one or more hydroxyl groups (preferably, the alkyl group is substituted at the end of the chain with a hydroxyl group), such as hydroxymethyl or 2-hydroxyethyl; preferably, (I′) represents a C₁-C₄ hydroxyalkyl (meth)acrylate such as hydroxyethyl acrylate; and
a₃″) of at least one monomer of formula (5) as defined previously, as well as its salts of organic or inorganic bases such as alkalis or alkaline-earth metals such as Na or K.

According to yet another particular embodiment, the different particle(s) preferably comprise an ethylenic copolymer (IB) resulting from the polymerization of:
a2″) of at least one monomer of formula (I′) H2C=C(R)—C(O)—O-[ALK-O]ₚ-R′, in which R represents a hydrogen atom or a (C₁-C₄)alkyl group such as methyl, R′ represents a (C₁-C₄)alkyl group such as methyl or ethyl, preferably a C1-C4 alkyl acrylate such as methyl acrylate; ALK represents a (C₁-C₆)alkylene group optionally substituted with at least one group chosen from Acid as defined previously and hydroxyl; preferably, ALK represents a (C₁-C₄)alkylene group such as ethylene, propylene, butylene or isobutylene; more preferentially, ALK represents an ethylene group; and p represents an integer greater than or equal to 1 and less than or equal to 100; and
a₃″) of at least one monomer of formula (5) as defined previously, as well as its salts of organic or inorganic bases such as alkalis or alkaline-earth metals such as Na or K, preferably acrylic acid.

In some embodiments, the core particles may include more than 50% of one monomer, and less than 50% of another monomer. For example, the core particles may include more than 75% 2-hydroxyethyl acrylate and less than 25% acrylic acid.

In some embodiments, one or more of the monomers is less than 100% neutralized.

According to this embodiment, the different particle(s) may preferably comprise ethylenic copolymers (IC) obtained from the polymerization of:
a′″₁) (C₁-C₄) (alkyl) acrylate (C₁-C₄) alkyl monomers, preferably of formula (I) defined above; and/or
a₂) poly[oxy(C₁-C₄)alkylene] (C₁-C₄)(alkyl)acrylate, and
a₃) ethylenic monomers comprising one or more carboxy, anhydride, phosphoric acid, sulfonic acid groups, preferably chosen from (1), (2), (3), and (4) as defined above, more particularly of formula (5)) as well as its salts of organic or inorganic bases such as alkalis or alkaline-earth metals such as Na or K and even more preferably acrylic acid, preferably ethylenic copolymers (IC) resulting from the polymerization of monomers a′″₁) and a₃), the monomers a′″₁) preferably denoting C1-C4 alkyl (meth) acrylates, in particular ethyl (meth) acrylate, methyl (meth) acrylate more preferably methyl acrylate, ethyl acrylate.

More preferably, the copolymers (IC) result from the polymerization of several different monomers a′″₁) and of monomers a3), even more preferably from ethyl (meth) acrylate with methyl (meth) acrylate and monomers a3), more particularly methyl acrylate with ethyl acrylate and monomers a3).

According to another embodiment, the dispersion comprises at least 2 types of different particles, preferably 2 types of different particles which are chosen from copolymers obtained by polymerization of monomers a₁) and a₃) as defined. previously, and b₂) polymers of ethylenic monomers comprising one or more carboxy, anhydride, phosphoric acid, sulfonic acid, preferably carboxy groups.

According to this embodiment, the copolymers are preferably chosen from copolymers (IC) as described above.

The polymers of ethylenic monomers b2) preferably denote the homopolymer (ID), more preferably a homopolymer of monomers chosen from (1), (2), (3), and (4) as defined above, more particularly of monomers of formula (5) as well as its salts of organic or inorganic bases such as alkalis or alkaline-earth metals such as Na or K and even more preferably of acrylic acid.

The ethylenic polymeric core particle(s) of the dispersion (have) preferably a number-average size is greater than 50 nm.

The final size of the core particles of structure (ID) included in the dispersion may preferably be greater than 50 nm. In particular, a number-average size ranging from 50 nm to 600 nm; more particularly ranging from 100 nm to 500 nm, even more particularly ranging from 150 nm to 400 nm.

The final size of the core particles of structure (IA), (IB), (IC) or (IE) included in dispersion (A) is preferably greater than 100 nm. In particular, a number-average size ranging from 100 nm to 100 μm; more particularly ranging from 500 nm to 10 μm.

The mean size of the ethylenic polymeric core particles is determined via conventional methods known to those skilled in the art. A Malvern brand NanoZS model laser particle size analyser (which is particularly suitable for submicron dispersions) makes it possible to measure the size distribution of these samples. The operating principle of this type of machine is based on dynamic light scattering (DLS), also known as quasi-elastic light scattering (QELS) or photon correlation spectroscopy (PCS). It is also possible to determine the particle size by transmission microscopy.

The sample is pipetted into a disposable plastic tank (four transparent faces, side length of 1 cm and volume of 4 mL) placed in the measuring cell. The data are analyzed on the basis of a cumulant fit method which leads to a monomodal particle size distribution characterized by an intensity-weighted mean diameter d (nm) and a size polydispersity factor Q. The results may also be expressed in the form of statistical data such as D10; D50 (median), D90 and the mode.

Other particle size techniques make it possible to obtain this type of information, such as analysis of the individual tracking of particles (Nanoparticle Tracking Analysis, NTA), laser scattering (LS), acoustic extinction spectroscopy (AES) spatial-filter Doppler velocimetry or image analysis.

Preferably, the monomers that are capable of forming the ethylenic core of the particle are chosen from monomers that are insoluble in the liquid hydrocarbon-based medium consisting of liquid hydrocarbon-based fatty substances. The insoluble monomers notably represent 100% by weight relative to the total weight of the monomers forming the polymeric core of the particle.

Preferably, the constituent monomers of the ethylenic core particles are chosen from monomers insoluble in the liquid hydrocarbon-based medium consisting of liquid fatty substance(s) hydrocarbon(s). The insoluble monomers represent in particular 80% to 100% by weight, preferably 90% to 100% by weight, more preferably 95% to 100% by weight, even more preferably 100% by weight, of the total weight of the monomers forming the ethylenic core particles particles(s).

### The Stabilizer(s)

The dispersion also includes one or more polymeric stabilizing agents (“stabilizers”). Preferably, only one type of stabilizer is used in the composition.

According to another particular embodiment, the stabilizer(s) are chosen from d) ethylenic homopolymers of (C3-C12)cycloalkyl (C1-C₆)(alkyl)acrylate monomers, preferably (C3-C12)cycloalkyl (meth)acrylate ethylenic homopolymers; more particularly ethylenic homopolymers derived from the polymerization of monomers of formula: H2C=C(R)—C(O)—O—R″ with R as defined previously, and R″ representing a (C5-C10)cycloalkyl group such as norbornyl or isobornyl, preferably isobornyl.

More particularly, the stabilizer(s) may consist of ethylenic polymers chosen from ethylenic homopolymers d) or d′) derived from the polymerization of monomers having the following formula: H2C=C(R)—C(O)—O—R′″, where R represents a hydrogen atom or a (C₁-C4)alkyl group such as methyl, and R′″ representing a (C5-C10)cycloalkyl group such as norbornyl or isobornyl, preferably isobornyl.

According to a particular embodiment, the stabilizer(s) may be chosen from e) copolymers of ethylenic monomers of e₁) (C₃-C₁₂)cycloalkyl (C₁-C₆)(alkyl)acrylate, and of e₂) (C₁-C₄)alkyl (C₁-C₄)(alkyl)acrylate, preferably copolymers of (C₃-C₁₂)cycloalkyl (meth)acrylate and of (C₁-C₄)alkyl (meth)acrylate.

According to another embodiment, the stabilizer(s) may be chosen from e′) ethylenic copolymers of e₁) (C₃-C₁₂)cycloalkyl (C₁-C₆)(alkyl)acrylate, and of e2) (C₁-C₄)alkyl (C₁-C₄) (alkyl)acrylate, preferably copolymers e1) of (C₃-C₁₂)cycloalkyl (meth)acrylate and e2) of (C₁-C₄)alkyl (meth)acrylate.

More particularly, the stabilizer(s) ii) are chosen from the ethylenic copolymers e) of monomers of formula (IV) and of monomers of formula (III):

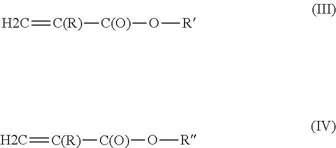

in which R, which may be identical or different, represents a hydrogen atom or a (C₁-C₄)alkyl group such as methyl; R′, which may be identical or different, represents a (C₁-C₄)alkyl group such as methyl or ethyl; and R″ represents a (C₅-C₁₀)cycloalkyl group such as norbornyl or isobornyl, preferably isobornyl.

According to another particular embodiment of the instant disclosure, R″ represents a (C₅-C₁₀)cycloalkyl group such as norbornyl or isobornyl, preferably isobornyl.

According to a particular embodiment, the stabilizer(s) may consist of ethylenic copolymers e) chosen from e₁) polymers of (C₃-C₁₂)cycloalkyl (C₁-C₆)(alkyl)acrylate monomers notably of formula (IV) and of formula (III) as defined previously.

More particularly, the stabilizer(s) may consist of ethylenic copolymers e) chosen from e′₁) norbornyl (meth)acrylate or isobornyl (meth)acrylate, preferably isobornyl (meth)acrylate, and e′2) methyl (meth)acrylate or ethyl (meth)acrylate.

According to another embodiment, the stabilizer(s) may be chosen from the ethylenic copolymers e) derived from the polymerization e₁) of a monomer of formula (IV) as defined previously and e₂) of two different monomers of formula (III) as defined previously.

Preferentially, the stabilizer(s) ii) are chosen from copolymers derived from the polymerization of er) one monomer of formula (IV) as defined previously notably chosen from isobornyl (meth)acrylate and e₂) of two different monomers of formula (III) as defined previously, notably different C₁-C₄ alkyl (meth)acrylates, preferably methyl and ethyl acrylate.

Particularly, the stabilizer may be chosen from d) homopolymers of (C3-C12)cycloalkyl (C₁-C₆)(alkyl)acrylate monomers; and e) statistical copolymers of e₁) (C3-C12)cycloalkyl (C₁-C₆)(alkyl)acrylate and e₂) (C₁-C₄)alkyl (C₁-C₄)(alkyl)acrylate with a weight ratio e₁/e₂ of greater than 4. Advantageously, said weight ratio ranges from 4.5 to 19. More advantageously, said weight ratio e₁/e₂ ranges from 5 to 15 and more preferentially said weight ratio ranges from 5.5 to 12.

More particularly, the stabilizer may be a polymer chosen from d′) isobornyl (meth)acrylate homopolymer and e) statistical copolymers of e₁′) isobornyl (meth)acrylate and of e₂′) C₁-C₄ alkyl (meth)acrylate preferably present in an isobornyl (meth)acrylate/C₁-C₄ alkyl (meth)acrylate weight ratio (e₁/e₂) of greater than 4. Advantageously, said weight ratio e₁′/e₂′ ranges from 4.5 to 19. Advantageously, said weight ratio e₁′/e₂′ ranges from 5 to 15 and more preferentially said weight ratio e₁′/e_{z}′ ranges from 5.5 to 14.

The stabilizing agent(s) as disclosed herein preferably comprise 80% to 100% by weight of monomer soluble in liquid hydrocarbon fatty substances iii), in particular from 85% to 95% by weight of soluble monomer, alone or as a mixture, relative to the total weight of monomers of the stabilizing agent (s). The stabilizer (co)polymer(s) may comprise particularly between 0% and 20% by weight, in particular between 5% and 15% by weight of monomer insoluble in liquid hydrocarbon fatty substances of the dispersion, alone or as a mixture relative to the total weight of monomers of the stabilizing agent(s).

Preferably, the stabilizer(s) and the particle(s) have a number-average molecular weight (Mn) of between 1000 and 1 000 000 g/mol, notably between 5000 and 500 000 g/mol and better still between 10 000 and 300 000 g/mol.

According to a particular embodiment, the dispersion may include from 5% to 40% by weight, in particular from 7% to 20% by weight, notably from 8% to 15% by weight and preferably from 9% to 13% by weight of (C3-C12)cycloalkyl (C1 C6)(alkyl)acrylate monomers d) or e1), relative to the total weight of polymers contained in said dispersion.

The stabilizer(s) may be present in the dispersion in an amount of between 0.01% and 30% by weight relative to the total weight of the dispersion, more particularly between 0.1% and 20% by weight, preferably between 0.5% and 10% by weight, more preferentially between 0.7% and 4.5% by weight relative to the total weight of the dispersion.

In some embodiments, the amount of stabilizer(s) may be between 0.05% and 30% by weight relative to the total weight of the dispersion without water, preferably between 0.2% and 20% by weight, notably between 2% and 15% by weight, more particularly between 2% and 10% by weight, more preferentially between 2.5% and 5% by weight relative to the total weight of the dispersion where the dispersion includes water.

In some embodiments, the amount of stabilizer(s) may be between 0.01% and 20% by weight relative to the total weight of said dispersion with water, preferably between 0.1% and 15% by weight, notably between 0.9% and 10% by weight, more particularly between 1% and 5% by weight, more preferentially between 2 and 3.5% by weight relative to the total weight of the dispersion where the dispersion includes water.

In some embodiments, the amount of stabilizer(s) may be are present in an amount of between 5% and 40% by weight relative to the total weight of the ethylenic core particles+stabilizer(s), more particularly between 8% and 30% by weight, preferably between 9.8% and 12.5% by weight relative to the total weight of the ethylenic core particles+stabilizer(s).

### The Hydrocarbon-Based Liquid Fatty Substance(s)

The dispersion of polymer particles according to the instant disclosure also includes one or more hydrocarbon-based liquid fatty substances in which said particles are dispersed.

In some embodiments, the hydrocarbon-based liquid fatty substance(s) are chosen from hydrocarbons, in particular alkanes, oils of animal origin, oils of plant origin, glycerides or fluorinated oils of synthetic origin, fatty alcohols, esters of fatty acids and/or of fatty alcohols, non-silicone waxes, and silicones; in particular, the liquid hydrocarbon-based fatty substance(s) are hydrocarbon-based oils, which are preferably volatile, or are a mixture of different volatile oils, more particularly isododecane.

The hydrocarbon-based liquid fatty substances are notably chosen from C₆-C₁₆ hydrocarbons or hydrocarbons comprising more than 16 carbon atoms and up to 50 carbon atoms, preferably between C₆ and C₁₆, and in particular alkanes, oils of animal origin, oils of plant origin, glycerides, fatty alcohols, fatty acid and/or fatty alcohol esters, and silicones.

In some embodiments, the fatty alcohols, fatty esters and fatty acids more particularly contain one or more linear or branched, saturated or unsaturated hydrocarbon-based groups comprising 6 to 50 carbon atoms, which are optionally substituted, in particular, with one or more (in particular 1 to 4) hydroxyl groups. If they are unsaturated, these compounds may comprise one to three conjugated or unconjugated carbon-carbon double bonds.

As regards the C₆-C₁₆ alkanes, they may be linear or branched, and possibly cyclic. Examples that may be mentioned include hexane, decane, undecane, dodecane, tridecane or isoparaffins, such as isohexadecane, isodecane or isododecane, and their mixtures such as the combination of undecane and tridecane such as for example CETIOL UT®, or mixtures of C₉-C₁₂ alkanes, preferably of natural origin, in particular linear or branched alkanes, C₉-C₁₂. This latter mixture is in particular known under the name INCI C₉-C₁₂ ALCANE, CAS 68608-12-8, VEGELIGHT SILK® marketed by BioSynthIs. This mixture of volatile, biodegradable, volatile oils obtained from coconut oil (viscosity is 0.9-1.1 cSt (40° C.)and a flash point at 65° C.).

The linear or branched hydrocarbons containing more than 16 carbon atoms may be chosen from liquid paraffins, petroleum jelly, liquid petroleum jelly, polydecenes, and hydrogenated polyisobutene such as Parleam®.

Among the hydrocarbon-based liquid fatty substances having an overall solubility parameter according to the Hansen solubility space of less than or equal to 20 (MPa)½, mention may be made of oils, which may be chosen from natural or synthetic, hydrocarbon-based oils, which are optionally branched, alone or as a mixture.

According to a very advantageous embodiment, the dispersion may include one or more liquid fatty substances which are one or more hydrocarbon-based oils. The hydrocarbon-based oil(s) may be volatile or non-volatile.

According to a preferred embodiment, the liquid hydrocarbon-based fatty substance(s) are hydrocarbon-based oils which are volatile or are a mixture of different volatile oils. That is, the oil component contains only volatile hydrocarbon oils.

According to a preferred embodiment, the fatty substance (s) are linear or branched hydrocarbon oils which are volatile in particular chosen from undecane, dodecane, isododecane, tridecane, and their mixture of different, volatile oils preferably comprising isododecane in the mixture, or a mixture of undecane and tridecane.

According to another particular embodiment, the liquid fatty substance (s) are a mixture of a volatile hydrocarbon oil and a non-volatile hydrocarbon oil, the mixture of which preferably comprises dodecane or isododecane as volatile oil.

According to another advantageous embodiment, the fatty substance(s) may be a mixture of non-volatile oil(s) and volatile oil(s), where preferably the volatile oil(s) include undecane, dodecane, isododecane, and/or tridecane, more preferably isododecane

More preferably when the fatty substance(s) are a mixture of volatile and non-volatile oil, the amount of volatile oil is greater than the amount of non-volatile oil.

According to another particular embodiment, the hydrocarbon-based liquid fatty substance(s) are a mixture of a volatile oil and a non-volatile oil such as an isododecane/octyldodecanol mixture or an isododecane/isononyl isononanoate mixture.

The hydrocarbon-based oil may be chosen from: hydrocarbon-based oils containing from 8 to 14 carbon atoms, and notably:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade names Isopar or Permethyl;
linear alkanes, for instance n-dodecane (C₁₂) and n-tetradecane (C₁₄) sold by Sasol under the references, respectively, Parafol 12-97 and Parafol 14-97, and also mixtures thereof, the undecane-tridecane mixture, mixtures of n-undecane (C₁₁) and of n-tridecane (C₁₃) obtained in examples 1 and 2 of patent application WO 2008/155 059 from the company Cognis, and mixtures thereof;
hydrocarbon-based oils of plant origin such as triglycerides constituted of fatty acid esters of glycerol, the fatty acids of which may have chain lengths ranging from C4 to C24, these chains possibly being linear or branched, and saturated or unsaturated; these oils are notably heptanoic acid or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil or musk rose oil; shea butter; or else caprylic/capric acid triglycerides, for instance those sold by the company Stearinerie Dubois or those sold under the names Miglyol 810®, 812® and 818®;
synthetic ethers containing from 10 to 40 carbon atoms;
linear or branched hydrocarbons of mineral or synthetic origin, such as petroleum jelly, polydecenes, hydrogenated polyisobutene such as Parleam®, squalane and liquid paraffins, and mixtures thereof,
esters such as oils of formula RIC(O)—O—R2 in which R1 represents a linear or branched fatty acid residue including from 1 to 40 carbon atoms and R2 represents a, notably branched, hydrocarbon-based chain containing from 1 to 40 carbon atoms, on condition that R1+R2³ 10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2 octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters;
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2 undecylpentadecanol.

In addition to the liquid hydrocarbon-based fatty substance, the dispersion may include a silicone oil. If the silicone oil is in the dispersion, it is preferably in an amount which does not exceed 10% by weight relative to the weight of the dispersion, more particularly in an amount of less than 5% and more preferentially 2%.

In particular, the dispersion may include at least one liquid hydrocarbon-based fatty substance iii) chosen from:
plant oils formed by fatty acid esters of polyols, in particular triglycerides, such as sunflower oil, sesame oil, rapeseed oil, macadamia oil, soybean oil, sweet almond oil, beauty-leaf oil, palm oil, grapeseed oil, corn oil, arara oil, cottonseed oil, apricot oil, avocado oil, jojoba oil, olive oil or cereal germ oil;
linear, branched or cyclic esters containing more than 6 carbon atoms, notably 6 to 30 carbon atoms; and notably isononyl isononanoate;
and more particularly the esters of formula Rd-C(O)—O—Re in which Rd represents a higher fatty acid residue including from 7 to 19 carbon atoms and Re represents a hydrocarbon-based chain including from 3 to 20 carbon atoms, such as palmitates, adipates, myristates and benzoates, notably diisopropyl adipate and isopropyl myristate;
hydrocarbons and notably volatile or non-volatile, linear, branched and/or cyclic alkanes, such as C5-C60 isoparaffins, which are optionally volatile, such as isododecane, Parleam (hydrogenated polyisobutene), isohexadecane, cyclohexane or Isopars; or else liquid paraffins, liquid petroleum jelly, or hydrogenated polyisobutylene; notably isododecane;
ethers containing 6 to 30 carbon atoms;
aliphatic fatty monoalcohols containing 6 to 30 carbon atoms, the hydrocarbon-based chain not including any substitution groups, such as oleyl alcohol, decanol, dodecanol, octadecanol, octyldodecanol and linoleyl alcohol; notably octyldodecanol;
polyols containing 6 to 30 carbon atoms, such as hexylene glycol; and
their mixtures, such as the combination of undecane and tridecane such as for example CETIOL UT®, preferably isododecane, or mixtures of linear or branched C₈-C₁₀ fatty acid esters and C₁₂ fatty alcohol C₆-C₁₈ alkanes resulting from the hydrogenation/complete reduction of mixtures of fatty acids obtained from Cocos Nucifera oil (coconut), in particular dodecane such as mixtures of cococaprylate/caprate and dodecane, there may be mentioned those of the INCI name coconut alkanes (and) Coco-caprylate/caprate marketed under the name VEGELIGHT 1212LC® by Grant Industries; or mixtures of C₉-C₁₂ alkanes, the chains of which comprise 9 to 12 carbon atoms, preferably linear or branched, C₉-C₁₂ alkanes, in particular comprising dodecane, there may be mentioned the mixture of oil from INCI name C₉-C₁₂ ALKANE, VEGELIGHT SILK® marketed by BioSynthIs.

Advantageously, the hydrocarbon-based liquid fatty substance(s) may be apolar, i.e., formed solely of carbon and hydrogen atoms.

Preferably, the dispersion may include at least one apolar liquid hydrocarbon-based fatty substance iii) preferably chosen from:
linear or branched C8-C30, in particular C10-C20 and more particularly C10-C16 alkanes, which are volatile or non-volatile, preferably volatile;
non-aromatic cyclic C5-C12 alkanes, which are volatile or non-volatile, preferably volatile; and
mixtures thereof.

The liquid hydrocarbon-based fatty substance(s) may preferably be chosen from hydrocarbon-based oils containing from 8 to 16 carbon atoms, in particular containing from 10 to 14 carbon atoms, which are preferably volatile, more particularly the apolar oils, as disclosed herein.

Among the branched C8-C16 and notably C10-C14 alkanes that are suitable for use as liquid hydrocarbon-based fatty substances in the dispersion, mention may be made of:
isoalkanes of petroleum origin (also known as isoparaffins), such as isododecane (also known as 2,2,4,4,6-pentamethylheptane), isodecane and, for example, the oils sold under the Isopar or Permethyl trade names,
linear alkanes, for instance n-dodecane (C12) and n-tetradecane (C14) sold by Sasol under the references, respectively, Parafol 12-97 and Parafol 14-97, and also mixtures thereof, the undecane-tridecane mixture, mixtures of n-undecane (C11) and of n-tridecane (C13) from the company Cognis, and
mixtures thereof.

Preferentially, the liquid hydrocarbon-based fatty substance(s) iii) may be apolar, more particularly isododecane.

According to another advantageous embodiment, the hydrocarbon-based liquid fatty substance(s) are a mixture of non-volatile and volatile oil; preferably, the mixture comprises isododecane as volatile oil or a mixture of oils, notably of undecane and tridecane or else isononyl isononanoate or octyldodecanol.

Preferably, the liquid hydrocarbon-based fatty substance(s) may be present in the dispersion of the invention in an amount of between 1% and 25% by weight, more preferentially between 2.5% and 20% by weight, even more preferentially between 4% and 15% and preferentially between 10% and 11.2% by weight relative to the total weight of the dispersion, where the dispersion may include water.

According to a particular embodiment of the invention, the liquid hydrocarbon-based fatty substance(s) may be present in the dispersion of the invention in an amount of between 1.5% and 30% by weight, more preferentially between 4% and 20% by weight, even more preferentially between 8% and 15% and notably between 12% and 13% by weight relative to the total weight of the dispersion, where the dispersion is free of water.

According to a particular embodiment of the invention, the one or more volatile hydrocarbons may be present from about 1 to about 85 wt. % based on the total weight of the cosmetic composition. In some instances, the cosmetic composition may include at least one or more volatile hydrocarbons chosen from isoparaffin, isohexadecane, isododecane, isodecane, undecane, tridecane, dodecane, isohexyl, isodecyl, neopentanoate, or a combination thereof. In at least one instance, isododecane and/or isoparaffins (e.g., C8-9 isoparaffin) are preferred. The cosmetic composition may be formulated to include volatile hydrocarbons that contain no silicon atoms.

The total amount of the volatile hydrocarbon may vary. In some embodiments, the cosmetic composition may include about 1 to about 85 wt. % based on the total weight of the cosmetic composition. In some cases, the total amount of volatile hydrocarbons is about 5 to about 85 wt %, about 10 to about 85 wt %, about 12 to about 85 wt %, about 14 to about 85 wt %, about 15 to about 85 wt %, about 1 to about 80 wt. %, about 5 to about 80 wt. %, about 10 to about 80 wt. %, about 15 to about 80 wt. %, about 10 to about 85 wt. %, about 20 to about 85 wt. %, about 20 to about 80 wt. %, about 30 to about 85 wt. %, based on the total weight of the cosmetic composition. In some cases, the total amount of volatile hydrocarbons is about 30 to about 80 wt. %, about 30 to about 75 wt. %; about 35 to about 85 wt. %, about 35 to about 80 wt. %, about 35 to about 75 wt. %; about 40 to about 85 wt. %, about 40 to about 80 wt. %, about 40 to about 75 wt. %; about 45 to about 85 wt. %, about 45 to about 80 wt. %, about 45 to about 75 wt. %; about 50 to about 85 wt. %, about 50 to about 80 wt. %, about 50 to about 75 wt. %; about 55 to about 85 wt. %, about 55 to about 80 wt. %, about 55 to about 75 wt. %; about 60 to about 85 wt. %, about 60 to about 80 wt. %, about 60 to about 75 wt. %; about 65 to about 85 wt. %, about 65 to about 80 wt. %, or about 65 to about 75 wt. %, including ranges and subranges thereof, based on the total weight of the cosmetic composition.

According to a particular embodiment of the invention, the mass ratio of the sum of the ingredients ([% by weight of ethylenic polymeric core particles+% by weight of polymeric stabilizing agents]/% by weight of fatty substance that is liquid at 20° C. and 1 atmosphere) is less than or equal to 3, more particularly the mass ratio is between 1 and 2.5, even more particularly between 1.5 and 2.4, preferentially between 1.7 and 2.2.

The dispersion may optionally include other components. For example, in some embodiments, the dispersion may include one or more polyols that are liquid at 20° C. and 1 atm, and/or may include water.

According to preferred embodiments, the hydrogel-in-oil dispersion is methyl acrylate/ethyl acrylate/acrylic acid (core)/isobornyl acrylate (stabilizer) (10/60/10/20) at 50% in isododecane.

The hydrogel-in-oil dispersion is preferably present in a content from about 15 to about 60% by weight, preferably from about 20 to about 50% by weight, preferably from about 25 to about 45% by weight, and preferably from about 30 to about 40% by weight, relative to the total weight of the composition containing the dispersion, including all ranges and subranges therebetween such as, for example, 15% to 30% by weight, 25% to 50% by weight, 18 to 52% by weight, etc. These weights reflect the weights of the hydrogel-in-oil dispersions including the oil(s) present. The amount of active (non-oil) component is preferably about 5% to about 50% of the dispersion, preferably about 10% to about 40%, preferably about 20% to about 35%, with about 33% being most preferred.

### Water

In some embodiments, the dispersion, notably the hydrogel, may comprises water in an amount of between 5% and 50% by weight relative to the total weight of the dispersion, more particularly between 7% and 48% by weight and preferentially between 9% and 46% by weight relative to the total weight of the dispersion.

In some embodiments, the weight ratio of the hydrocarbon-based liquid fatty substance(s)/the water is between 0.1 and 5, more particularly between 0.2 and 1, preferably between 0.3 and 0.7, more preferentially between 0.4 and 0.6.

The water that is suitable for use in the instant disclosure may be tap water, distilled water, spring water, a floral water such as cornflower water and/or a mineral water such as Vittel water, Lucas water or La Roche Posay water and/or a thermal water.

For purposes of compositions of the present invention containing the hydrogel-in-oil dispersion, preferably compositions of the present invention comprise 5% or less water, preferably 4% or less water, preferably 3% or less water, preferably 2% or less water, and preferably 1% or less water, all weights based on the total weight of the composition.

According to preferred embodiments, compositions of the present invention comprise 2% or less additional water which is not present in the hydrogel-in-oil dispersion, preferably 1% or less of such additional water, preferably 0.5% or less of such additional water, preferably 0.25% or less of such additional water, preferably 0.1% or less of such additional water, and preferably no such additional water, all weights based on the total weight of the composition.

The dispersion may also comprise one or more water-miscible solvents, which may be present even if the dispersion does not contain water. In this sense, the dispersion can contain water and/or one or more water-miscible solvents. Similarly, compositions of the present invention can contain water and/or one or more water-miscible solvents.

According to the instant disclosure, the term “water-miscible solvent” denotes a compound that is liquid at room temperature and water-miscible (miscibility with water of greater than 50% by weight at 25° C. and atmospheric pressure). The water-miscible solvents that may be used in dispersion of the instant disclosure may also be volatile. Among the water-miscible solvents that may be used in the composition according to the invention, mention may notably be made of lower monoalcohols containing from 2 to 5 carbon atoms such as ethanol and isopropanol.

The water may also comprise any water-soluble or water-dispersible compound that is compatible with an aqueous phase, such as associated polyols, film-forming polymers, surfactants, and mixtures thereof. As with water-miscible solvents, it should be understood that compositions of the present invention can contain water and/or one or more polyols as discussed above.

The term “surfactant” means a “surface agent”, which is a compound that is capable of modifying the surface tension between two surfaces; surfactants are amphiphilic molecules, i.e. they contain two parts of different polarity, one lipophilic and apolar, and the other hydrophilic and polar. The surfactants may be nonionic, anionic, amphoteric or cationic active agents.

The term “polyol” means a compound which is liquid at 20° C. and 1 atm, comprising at least 2 hydroxyl groups, preferably between 2 and 10 hydroxyl groups (OH), and comprising at least one carbon atom. Preferably, the polyol(s) are chosen from compounds comprising at least 2 OH groups, preferably between 2 and 8 OH groups, more preferentially 2 or 3 OH groups, even more preferentially 3 OH groups, and comprising a linear or branched, cyclic or acyclic, saturated or unsaturated hydrocarbon-based chain comprising from 1 to 10 carbon atoms, in particular between 2 and 8 carbon atoms. Suitable examples of polyols include glycerin and diols such as propanediol and butanediol.

In some embodiments, the dispersion may not comprise more than 3% by weight of surfactants relative to the total weight of the dispersion, preferentially not more than 2% by weight of surfactants relative to the total weight of the dispersion, more particularly not more than 1% by weight of surfactants relative to the total weight of the dispersion; even more preferentially, the composition does not comprise more than 0.5% by weight of surfactants relative to the total weight of the dispersion, and better still the mixture does not comprise any surfactant.

In some embodiments, the dispersion includes 10-20% of the ethylenic polymeric core particles, 15-35% of the hydrocarbon-based liquid fatty substance(s) 10-30% of the polymeric stabilizer, and 40-60% of water, by weight relative to the total weight of the dispersion.

### Film Forming Component Containing Glycerolated Silicone Resin

In accordance with the present invention, compositions comprising at least one glycerolated silicone resin are provided.

Preferably, compositions of the present invention comprise a film forming component comprising at least one glycerolated silicone resins, at least two glycerolated silicone resins, at least three glycerolated silicone resins, at least four glycerolated resins, etc. Accordingly, film forming components of the present invention can contain, for example, any number of glycerolated silicone resins such as from 1 to 10 such resins, 1-5 such resins, 1-3 such resins, etc. with a single glycerolated silicone resin being preferred.

The term “glycerolated silicone resin” is understood to mean a resin which comprises, in its chemical structure, one or more monoglycerol or polyglycerol group(s).

According to a specific embodiment of the invention, the glycerolated silicone resin or resins is (are) preferably present in a content as active material ranging from 0.1% to 40% by weight, with respect to the total weight of the composition, preferably ranging from 0.2% to 30% by weight and more preferably from 0.5% to 15% by weight, with respect to the total weight of the composition, including all ranges and subranges therebetween such as 1% to 40%, 1% to 10%, 2.5% to 20%, 5% to 12.5%, etc.

The glycerolated silicone resin(s) according to the invention are preferably chosen from those of following formula (1):

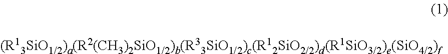

in which
   each R¹, which are identical or different, is an alkyl, aryl or aralkyl group of 1 to 30 carbon atoms, or a group substituted by a halogen, a group substituted by an amino or a group substituted by a carboxyl of the latter;
   each R² is a mono- or polyglycerol group of following general formula (2)

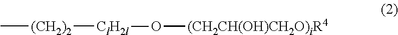

in which
   R⁴ is a substituted or unsubstituted monovalent hydrocarbon group or a hydrogen atom, and
   the indices 1 and i are numbers which satisfy the conditions 0≤1≤15 and 0<i≤5,
   each R³ is an identical or different group of general formula (3), of general formula (4), of general formula (5) or of general formula (6) below

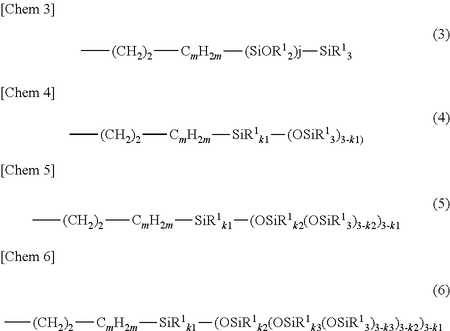

where
   each R¹, which are identical or different, is an alkyl, aryl or aralkyl group of 1 to 30 carbon atoms, or a group substituted by a halogen, a group substituted by an amino or a group substituted by a carboxyl of the latter;
   the indices m, j and k₁ to k₃ are numbers which satisfy the conditions

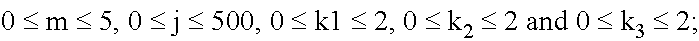

the indices a, b, c, d, e and f are numbers which satisfy the conditions 0≤a≤400, 0<b≤200, 0≤c≤400, 0≤d≤320, 0≤e≤320, 0<f≤1000 and 0.5≤(a+b+c)/f≤1.5.

The glycerolated silicone resins according to the invention are described in the patent application US20200332065A1 of SHIN-ETSU.

According to a specific embodiment, the glycerolated silicone resin(s) of formula (1) as defined above are chosen from those for which
the indices b and c satisfy the conditions 0<b≤30 and 0≤c≤30;
the index i in the general formula (2) of the monoglycerol or polyglycerol group R2 is a number which satisfies the condition 0<i≤3.

According to a specific embodiment, the glycerolated silicone resin(s) of formula (1) are in the solid form at 25° C. when the index c satisfies the condition 0<c≤400 and R3 is a group of general formula (3) where the index j satisfies the condition 0≤j≤10.

According to a specific embodiment, the glycerolated silicone resin(s) exhibit a weight-average molecular weight ranging from 1000 to 100 000.

The glycerolated silicone resin(s) according to the invention are amphiphilic, that is to say exhibit two parts of different polarities. In general, one is lipophilic (soluble or dispersible in an oily phase). The other is hydrophilic (soluble or dispersible in water). They are characterized by the value of their HLB (Hydrophilic-Lipophilic Balance), the HLB being the ratio of the hydrophilic part to the lipophilic part in the molecule. The term HLB is well known to a person skilled in the art and is described, for example, in “The HLB System. A Time-Saving Guide to Emulsifier Selection” published by ICI Americas Inc., 1984). The value of the HLB of the glycerolated silicone resins according to the invention preferably varies from 0.1 to 15 according to the Griffin method.

The glycerolated silicone resin(s) according to the invention can be obtained by a preparation process comprising the stage of hydrosilylation

A) of a silicone resin containing a hydrosilyl group of formula (7) below

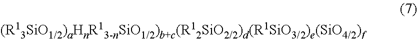

in which:
   each R1, which are identical or different, is an alkyl, aryl or aralkyl group of 1 to 30 carbon atoms, or a group substituted by a halogen, a group substituted by an amino or a group substituted by a carboxyl of the latter;
   the indices a, b, c, d, e and f are numbers which satisfy the conditions 0≤a≤400, 0<b≤200, 0≤c≤400, 0≤d≤320, 0≤e≤320, 0<f≤1000 and 0.5≤(a+b+c)/f≤1.5;
   n is a number which satisfies a condition 1≤n≤3, with
B) one or more compounds which are chosen from the compounds terminated by an alkenyl group of general formulae (8), (9), (10), (11) and (12) below]

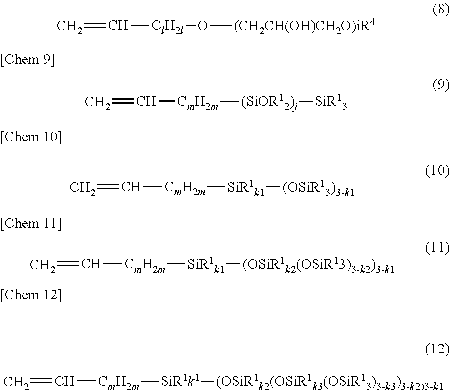

where
   R+is a substituted or unsubstituted monovalent hydrocarbon group or a hydrogen atom,
   the indices 1 and i are numbers which satisfy the conditions 0≤1≤15 and 0<i≤5;
   the indices m, j and k₁ to k₃ are numbers which satisfy the conditions 0≤m≤5, 0≤j≤500, 0≤k₁≤2, 0≤k₂≤2 and 0≤k₃≤2; said silicone resin containing a hydrosilyl group of formula (7) which reacts with at least one compound in the formula (8).

The hydrosilylation reaction is carried out in the presence, for example, of a platinum or rhodium catalyst. The preferred ranges for b, c, d, e, f, R⁴, 1, m, i, j and k₁ to k₃ are as defined above.

### Process for the Preparation of the Glycerolated Silicone Resin

A specific example of process for the preparation of the glycerolated silicone resin according to the invention is described below.

As mentioned above, the glycerolated silicone resin according to the invention can be obtained by the stage of hydrosilylation

(A) of a silicone resin containing a hydrosilyl group of mean formula (7) below:

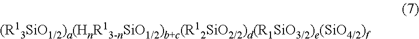

in which
   each R1 is an identical or different alkyl, aryl or aralkyl group of 1 to 30 carbon atoms, or a group substituted by a halogen, a group substituted by an amino or a group substituted by a carboxyl of the latter;
   the indices a, b, c, d, e and f are numbers which satisfy the conditions 0≤a≤400, 0<b≤200, 0≤c≤400, 0≤d≤320, 0≤e≤320, 0<f≤1000 and 0.5≤(a+b+c)/f≤1.5;
   n is a number which satisfies the condition 1≤n≤3, with
(B) one or more compounds which are chosen from the compounds terminated by an alkenyl group of general formulae (8), (9), (10), (11) and (12) below

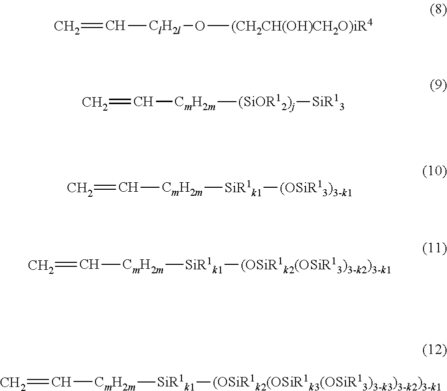

where
   R⁴ is a substituted or unsubstituted monovalent hydrocarbon group or a hydrogen atom,
   the indices 1 and i are numbers which satisfy the conditions 0≤1≤15 and 0<i≤5;
   the indices m, j and k₁ to k₃ are numbers which satisfy the conditions 0≤m≤5, 0≤j≤500, 0≤k₁≤2, 0≤k₂≤2 and 0≤k₃≤2; said comprise a compound of general formula (8).

The organosilicon resin containing hydrosilyl groups of mean composition formula (7) and the compound having terminal alkenyl groups of general formula (8), (9), (10), (11) or (12) are mixed in a molar ratio, expressed as hydrosilyl groups/terminal unsaturated groups, which is preferably from 0.5 to 2.0 and more preferentially from 0.8 to 1.2.

The addition reaction is preferably carried out in the presence of a platinum or rhodium catalyst. Specific examples include chloroplatinic acid, chloroplatinic acid modified by an alcohol and chloroplatinic acid/vinylsiloxane complexes. When an excessive amount of catalyst is included, a discoloration of the sample is produced, and thus the amount of platinum or of rhodium is preferably 50 ppm or less and more preferably 20 ppm or less.

In addition, if necessary, the addition reaction can be carried out in the presence of an organic solvent. Mention may be made, among the examples of organic solvent, of cyclic organopolysiloxanes, such as octamethylcyclotetrasiloxane, decamethylcyclopentasiloxane and dodecamethylcyclohexasiloxane; aromatic hydrocarbons, such as toluene and xylene; solvents of ketone type, such as acetone, methyl ethyl ketone, diethyl ketone and methyl isobutyl ketone; aliphatic hydrocarbons, such as hexane, heptane, octane and cyclohexane; and aliphatic alcohols, such as methanol, ethanol, 1-propanel, 2-propanol, 1-butanol, 2-methyl-1-propanol, 2-butanol, 2-methyl-2-propanol, 1-pentanol, 2-methylbutanol, 2-pentanol, 1-hexanol, 2-methylpentanol, 1-heptanol, 1-octanol, 1-nonanol, 1-decanol, phenol, benzyl alcohol, ethylene glycol and 1,2-propylene glycol. From the viewpoint of the reactivity, ethanol, 1-propanol and 2-propanol are preferred.

The amount of solvent used is preferably from 1% to 80% and more preferably from 5% to 50% of the overall reaction system. In the above range, the reaction system is kept uniform and the reaction takes place efficiently.

The conditions of the addition reaction are not particularly limited, although heating at reflux at a temperature of between 50 and 150° C., in particular between 80 and 120° C., for approximately 1 to 10 hours is preferred.

After the addition reaction, the stage of removal of the rhodium or platinum catalyst used with activated carbon can be included. The amount of activated carbon used is preferably from 0.001% to 5.0% and in particular from 0.01% to 1.0% of the overall system. The discoloration of the sample can be better suppressed by fixing the amount of activated carbon in this range.

After the addition reaction, if necessary, the stage of removal of the remaining hydrosilyl groups can be included. In particular in the cases where use in applications such as cosmetic preparations is anticipated, there exists a possibility of these hydrosilyl groups becoming deactivated over time due to dehydrogenation reactions, which presents a problem from the viewpoint of safety. It is thus preferable to include a stage of maintenance of the hydrosilyl groups.

An example of stage of removal of the hydrosilyl groups is the process of hydrolysis of the unreacted hydrosilyl groups by the addition of a basic catalyst, such as an alkali metal carbonate, an alkali metal bicarbonate or an alkali metal hydroxide, and then neutralization by the addition of an amount of acid catalyst equal to the molar equivalent of the basic catalyst. Specific examples of the basic catalyst comprise strong basic catalysts, such as lithium hydroxide, sodium hydroxide, potassium hydroxide, calcium hydroxide and barium hydroxide; and weak basic catalysts, such as sodium carbonate, calcium carbonate and sodium bicarbonate. From the viewpoint of the promotion of the dehydrogenation reaction, the use of a strong basic catalyst is particularly preferred, sodium hydroxide being particularly preferred. Mention may be made, among acid catalysts, of hydrochloric acid, sulfuric acid, sulfurous acid, fuming sulfuric acid, oxalic acid, p-toluenesulfonic acid, methanesulfonic acid, trifluoroacetic acid, trifluoromethanesulfonic acid, phosphoric acid, formic acid, acetic acid, propionic acid, benzoic acid and citric acid. In general, instead of using the acid alone or the base alone, it is preferable to use them with water and to heat them to a temperature which is not greater than the boiling point of water.

After the addition reaction, if necessary, the stage of deodorization to reduce the odor can be included. When use in applications such as cosmetic preparations in particular is anticipated, because the product acquires an odor with time, it is preferable to include a stage of deodorization. The mechanism for the deodorization of ordinary silicones modified by polyethers can be explained as follows. When an addition reaction between a polyether etherified by allyl groups and a hydropolyorganosiloxane is carried out in the presence of a platinum catalyst, the allyl groups rearrange internally in the form of side reactions, forming a polyether etherified by propenyl groups. This propenyl-etherified polyether has no reactivity with the polyorganosiloxane hydrogen and thus remains in the system as an impurity. It is believed that when water acts on this propenyl-etherified polyether, the propenyl ether hydrolyzes, giving rise to propionaldehyde, which gives off an unpleasant odor. It is known that the above hydrolysis reaction is also promoted in the presence of an acid catalyst. Consequently, when the silicone modified by a polyether is used in a water-based cosmetic preparation, due to the oxidative deterioration of the polyether, the preparation tends to become acidic with time, promoting the hydrolysis reaction described above and bringing about the appearance of a bad odor.

Typical examples of the stage of deodorization comprise two approaches. The first is that in which, by adding an acid catalyst to the solution after the addition reaction, any propenyl ether remaining in the system is hydrolyzed and the propionaldehyde which is formed is removed by strip purification (JP No. 2137062).

Specific examples of the acid catalyst used in the first approach comprise hydrochloric acid, sulfuric acid, sulfurous acid, fuming sulfuric acid, oxalic acid, p-toluenesulfonic acid, methanesulfonic acid, trifluoroacetic acid, trifluoromethanesulfonic acid, phosphoric acid, formic acid, acetic acid, propionic acid, benzoic acid and citric acid. These acids are used in combination with water. In the case where it is necessary to remove the acid which has been used, it is preferable to use an acid having a low boiling point, such as hydrochloric acid, formic acid, acetic acid or trifluoroacetic acid. Likewise, from the viewpoint of the effectiveness of the treatment, it is preferable to use a strong acid, such as hydrochloric acid or trifluoroacetic acid.

The treatment temperature is preferably fixed at 80° C. or less in order to prevent the oxidation of the hydrophilic groups. The amount of acidic aqueous solution added is preferably fixed from 0.1% to 100%, with respect to the organosilicon resin modified by organic groups. The use of 5% to 30% is more preferred.

From the viewpoint of the productivity, the process consisting of adding an aqueous solution to the post reaction solution so as to regulate the pH at 7 or less and of carrying out a strip purification after stirring under heating is preferred. The purification of the strip can be carried out at normal temperature or under reduced pressure. The temperature conditions are preferably fixed at 120° C. or less. In order to efficiently purify the strip under these temperature conditions, it is preferable to carry out this operation under reduced pressure; when it is carried out at normal pressure, the operation is preferably carried out under a stream of inert gas, such as nitrogen or argon.

The second approach is that in which, by adding hydrogen to the solution after the addition reaction, the unsaturated double bonds are alkylated (subjected to a hydrogenation reaction) and the formation of propionaldehyde over time is controlled in a stable manner (U.S. Pat. No. 5,225,509; JP-A H07-330907).

The hydrogenation reactions comprise methods involving the use of hydrogen and methods involving the use of metal hydrides, and there also exist homogeneous reactions and heterogeneous reactions. These methods can be used alone but it is also possible to use in them combination. However, given the advantage that there is no trace of catalyst used in the product, a heterogeneous catalytic hydrogenation reaction using a solid catalyst is preferred.

The solid catalyst is, for example, nickel, palladium, platinum, rhodium, cobalt, chromium, copper, iron and others, in the uncombined form or in the compound form. In this case, it is not necessary to use a catalyst support. However, when a catalyst support is used, the support can, for example, be activated carbon, silica, silica/aluminum, aluminum or zeolite. These catalysts can be used alone but it is also possible to use them in combination. The preferred catalyst is Raney nickel, which is economically advantageous. As the Raney nickel is generally developed and used with an alkali, it is necessary to carefully measure the pH of the reaction system. Furthermore, the reaction system becomes weakly alkaline, which is particularly effective for the deodorization when the hydrolysis reaction is carried out with an acidic aqueous solution.

It is preferable to carry out the hydrogenation reaction at a pressure generally of between 1 and 100 MPa and between 50 and 200° C. The hydrogenation reaction can be carried out batchwise or continuously. When it is a noncontinuous process, the reaction time depends, for example, on the amount of catalyst and on the temperature but it is generally of between 3 and 12 hours. The hydrogen pressure can be adjusted to an appropriate fixed pressure. The final point of the hydrogenation reaction is the point at which the hydrogen pressure has ceased to change and it can thus be determined by carefully monitoring a manometer.

The amount of aldehyde included in the glycerolated silicone resin which has been purified by this acid treatment and this hydrogenation treatment can be fixed at 70 ppm or less, preferably at 20 ppm or less and more preferably at 10 ppm or less.

It is also possible to combine both types of abovementioned deodorization stages. In the approach which involves an acid treatment, the decomposition and the removal of the aldehyde compound is possible but, as there is a limit to the complete removal of the unsaturated double bonds, the formation of odorous aldehyde from that cannot be completely suppressed. In the approach which involves a hydrogenation reaction, by eliminating the unsaturated double bonds, it is possible to reduce the amount of aldehyde compound which is formed because of that. However, the aldehyde condensate which is formed with the condensation of a portion of the aldehyde remains in the system even after such a treatment has been carried out and the removal by strip purification is also difficult. Consequently, by alkylating the unsaturated double bonds which remain when the solution, following the addition reaction, is subjected to hydrogenation, and by subsequently decomposing the aldehyde condensate in the system by adding an acid catalyst, complete deodorization is possible (WO2002/05588).

The weight-average molecular weight of the glycerolated silicone resin of mean formula (1) preferably varies from 1000 to 100 000; from the viewpoint of the performance qualities and of the ease of the operations, such as the filtration, the weight-average molecular weight preferentially varies from 3000 to 50 000. Here and subsequently, the weight-average molecular weight can be determined as the polystyrene-equivalent weight-average molecular weight in gel permeation chromatography (GPC).

The glycerolated silicone resin according to the invention is in a form at 25° C. which can be solid or liquid; from the viewpoint of the formability of the film, it is preferably solid.

In particular, the glycerolated silicone resin according to the invention of formula (1) for which the indices b and c satisfy the conditions 0<b≤30 and 0≤c≤30, the index i in the general formula (2) is a number which satisfies the condition 0<i≤3 and the index j in the general formula (3) satisfies the condition 0≤j≤10 is the form of a solid at 25° C. and preferably exhibits a weight-average molecular weight which preferably varies from 1000 to 100 000 and more preferentially from 3000 to 50 000.

The glycerolated silicone resins according to the invention have a hydrophilic-lipophilic balance (HLB), as determined by the Griffin formula, preferably of 0.1 to 15 and more preferably of 1.0 to 8.0.

According to a preferred form, the composition of the invention comprises at least one glycerolated silicone resin in the formula (1) of the (3-Glyceroxypropyl) Dimethylsiloxy Trimethylsiloxysilicate type corresponding to the following formula (21):

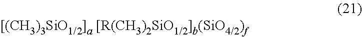

where
   R denotes the 3-glyceroxypropyl group of structure

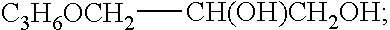

the indices a, b and f are numbers which satisfy the conditions 0≤a≤400, 0<b≤30, 0<f≤1000 and 0.5≤(a+b)/f≤1.5.

According to a particularly preferred form, the glycerolated silicone resin of the (3-Glyceroxypropyl) Dimethylsiloxyl Trimethylsiloxysilicate type of formula (21) is in the form of a solution in at least one volatile oil.

The term “volatile oil” is understood to mean, within the meaning of the invention, any oil capable of evaporating on contact with the skin in less than one hour, at ambient temperature and atmospheric pressure. The volatile oil is a volatile cosmetic compound, liquid at ambient temperature, having in particular a non-zero vapor pressure, at ambient temperature and atmospheric pressure, in particular having a vapor pressure ranging from 2.66 Pa to 40 000 Pa, in particular ranging from 2.66 Pa to 13 000 Pa and more particularly ranging from 2.66 Pa to 1300 Pa.

The volatile oil in accordance with the invention can be chosen from the group constituted of hydrocarbon oils, silicone oils and their mixtures.

The term “hydrocarbon oil” is understood to mean an oil containing predominantly hydrogen and carbon atoms and optionally one or more functions chosen from hydroxyl, ester, ether and carboxyl functions.

Within the meaning of the present invention, the term “silicone oil” denotes an oil comprising at least one Si—O group and more particularly an organopolysiloxane.

The volatile hydrocarbon oils which can be used in the compositions according to the invention can be chosen from branched C8-C16 alkanes.

Mention may in particular be made, as C8-C16 isoalkanes of petroleum origin (also known as isoparaffins), such as isododecane (also known as 2,2,4,4,6-pentamethylheptane), isodecane, isohexadecane and, for example, the oils sold under the Isopar® or Permethyl® tradenames. More preferentially, isododecane will be used.

Mention may be made, by way of example of volatile silicone oil which can be used in the invention, of volatile silicone oils, such as volatile linear or cyclic silicone oils, in particular those having a viscosity of 2 to 8 centistokes (2.10⁻⁶ to 8.10⁻⁶ m²/s) and containing in particular from 2 to 7 silicon atoms, these silicones optionally comprising alkyl or alkoxy groups containing from 1 to 10 carbon atoms. Mention may in particular be made, as volatile silicone oils which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetra-siloxane and dodecamethylpentasiloxane; and their mixtures. More preferentially, decamethylcyclopentasiloxane (D5) will be used.

According to a particularly preferred form, the glycerolated silicone resin of the (3-Glyceroxypropyl) Dimethylsiloxy Trimethylsiloxysilicate type of formula (21) is in the form of the solution comprising 49.5% by weight of active material in isododecane, such as the product manufactured under the tradename X-25-9138A® by SHIN ETSU with a weight-average molecular weight of 11 000.

According to a preferred form, in order to improve the wear property of the composition of the invention with regard to sebum, the composition of the invention comprises at least one glycerolated silicone resin and at least one nonglycerolated silicone resin in a ratio by weight of the amount of glycerolated silicone resin to the amount of nonglycerolated silicone resin of greater than or equal to 0.8 and more preferentially of greater than or equal to 1.0.

Preferably, the weight ratio of active material (non-oil) in the hydrogel-in-oil dispersion to glycerolated silicone resin is from about 3:1 to about 1:3, from about 2:1 to about 1:2, from about 1.5 to 1 to about 1:1.5, and about 1:1, including 12:5 to 5:12, 12:10 to 10:12, 12:5 to 5:10 (that is, 1:2), 12:10 to 5:10 (that is, 1:2). 10:5 (that is, 2:1) to 5:12, etc, including any combination of ratios identified in the examples of the present specification.

### Colorant

According to preferred embodiments, compositions of the present invention further comprise at least one colorant in an amount effective to provide visible color to a keratinous material to which the composition has been applied. However, compositions of the present invention can also be “free” or “substantially free” or “devoid of” colorant as discussed above.

The colorants that may be incorporated into the composition may include pigments, dyes, such as liposoluble dyes, nacreous pigments, and pigments with special effects, and/or pearlescent agents.

Representative liposoluble dyes which may be used according to the present invention include Sudan Red, DC Red 17, DC Green 6, B-carotene, soybean oil, Sudan Brown, DC Yellow 11, DC Violet 2, DC Orange 5, annatto, and quinoline yellow. The liposoluble dyes, when present, generally have a concentration ranging up to 40% by weight of the total weight of the composition, such as from 0.0001% to 30%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.0001% to 40%, preferably from 0.001% to 30%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

As used herein, the term “pigment” refers to any pigment that gives color to keratin materials, of synthetic or natural origin, the solubility of the pigments in water at 25° C. and at atmospheric pressure (760 mmHg) being less than 0.05% by weight and preferably less than 0.01%.

As used herein, the term “lake” refers to dyes adsorbed onto insoluble particles, the assembly thus obtained remaining insoluble during use. The inorganic substrates onto which the dyes are adsorbed are, for example, alumina, silica, calcium sodium borosilicate or calcium aluminum borosilicate and aluminum. Among the organic dyes, mention may be made of cochineal carmine.

As used herein, the term “pigments with special effects” refers to pigments that generally create a colored appearance (characterized by a certain shade, a certain vivacity and a certain level of luminance) that is non-uniform and that changes as a function of the conditions of observation (light, temperature, angles of observation, etc.). They thereby differ from colored pigments, which afford a standard uniform opaque, semi-transparent or transparent shade. In some embodiments, the pigment with special effects may include those with a low refractive index, such as fluorescent, photochromic or thermochromic pigments, and those with a higher refractive index, such as nacres or glitter flakes.

In some embodiments, the colorants may be present in the composition in a concentration ranging up to 50% by weight of the total weight of the composition, such as from 0.0001% to 40%, and further such as from 0.001% to 30%, including all ranges and subranges therebetween. In the case of certain products, the pigments, including nacreous pigments, may, for example, represent up to 50% by weight of the composition such as, for example, from 6% to 50% of the composition.

### Additional Additives

The composition of the invention can also comprise any additive usually used in the field under consideration. For example, additional film forming agents, waxes, dispersants such as poly(12-hydroxystearic acid), sunscreens, preserving agents, fragrances, fillers, antioxidants, vitamins, neutralizing agents, cosmetic and dermatological active agents, moisturizers, silicone elastomers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002). However, it is to be understood that preferred embodiments of the present invention include compositions which are “free,” substantially free” or “devoid” of the ingredients discussed in this paragraph such as additional film forming agents, waxes and/or sunscreens, etc.

If present, active agents can be present in compositions of the present invention in an amount preferably of at least 3% by weight, at least 5% by weight, at least 7% by weight, at least 10% by weight, and at least 15% by weight up to amounts such as 50% by weight, such as 40% by weight, such as 30% by weight, and such as 20% by weight, including all ranges and subranges therebetween including 3% to 50%, 5% to 40% 10% to 20%, 7% to 30%, etc., all weights based on the total weight of the composition. Suitable active agents for optional inclusion into the compositions of the present invention include, but are not limited to hydrophilic active agents such as, for example,
a moisturizing agent, such as a polyol (as defined and discussed above in connection with water-miscible solvents), sugars, urea and its derivatives, such as in particular hydroxyalkylurea, and mixtures thereof;
a desquamating agent, which may be a compound capable of acting either directly on desquamation by promoting exfoliation, such as B-hydroxy acids, in particular salicylic acid and its derivatives (including 5-n-octanoylsalicylic acid); a-hydroxy acids, such as glycolic acid, citric acid, lactic acid, tartaric acid, malic acid or mandelic acid; urea; gentisic acid; oligofucoses; cinnamic acid; extract of Saphora japonica, resveratrol and certain jasmonic acid derivatives; or acting on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; aminosulfonic compounds and in particular (N-2-hydroxyetbylpiperazine-N-2-ethane)sulfonic acid (HEPES); 2-oxothiazolidine-4-carboxylic acid (procysteine) derivatives; a-amino acid derivatives of the type such as glycine (as described in EP-0 852 949 and sodium methylglycinediacetate sold by BASF under the trade name Trilon M); honey: sugar derivatives such as O-octanoyl-6-D-maltose and N-acetylglucosamine;
a humectant;
an anti-aging agent, which may include, for example, one or more of C-beta-D-xylopyranoside-2-hydroxypropane (Pro-Xylane), retinol, peptides, caffeine, and other components that provide improvement to skin texture, any other suitable soluble/dispersible targeted active ingredient, and combinations thereof;
a mattifying agent, which may include, but is not limited to, mattifying fillers such as, for example, talc, silica, silicone elastomers, and polyamides, and waxes such as, for example, beeswax and copernicia cerifera (carnauba) wax;
a healing agent;
an antimicrobial agent, non-limiting examples of which include 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, micronazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoylglycine, caprylyl glycol, 10-hydroxy-2-decanoic acid, dichlorophenylimidazole dioxolane and its derivatives described in patent WO 93/18743, farnesol and phytosphingosines, and mixtures thereof;
a water-soluble texture modifier;
a pigment modifying agent and/or skin lightening agents, such as double-stranded RNA oligonucleotides are useful for decreasing tyrosinase expression. Mention may also be made of ceramides, vitamin C and derivatives thereof, in particular vitamin CG, CP and 3-( )ethyl vitamin C, alpha- and beta-arbutin, ferulic acid, kojic acid, resorcinol and derivatives thereof, calcium D-pantetheine sulphonate, lipoic acid, ellagic acid, vitamin B3, phenylethyl resorcinol, for instance Symwhite 377® from the company Symrise, a kiwi fruit (Actinidia chinensis) juice sold by Gattefosse, an extract of Paeonia suffructicosa root, such as the product sold by the company Ichimaru Pharcos under the name Botanpi Liquid B®, an extract of brown sugar (Saccharion officinarunt), such as the extract of molasses sold by the company Taiyo Kagaku under the name Molasses Liquid, a mixture of undecylenic acid and undecylenoyl phenyl alanine, such as Sepiwhite MSH® from Seppic;
a vitamin such as vitamin B3, vitamin C, and/or derivatives thereof;
antioxidants may include tocopherol and esters thereof, in particular tocopheryl acetate; EDTA, ascorbic acid and derivatives thereof, in particular magnesium ascorbyl phosphate and ascorbyl glucoside; chelating agents, such as BHT, BHA, N,N′-bis(3,4,5-trimethoxybenzyl)ethylenediamine and its salts, and mixtures thereof,
ultraviolet (UV) filter may include, e.g., an aminobenzoic acid derivative, a dibenzoylmethane derivative, a salicylic acid derivative, a cinnamic derivative, a β,β diphenylacrylate derivative, a benzophenone derivative, benzylidene camphor derivative, and mixtures thereof. Mention may be made especially of ethylhexyl methoxycinnamate sold under the tradename UVINUL MC 80® by the company BASE, of ethylhexyl salicylate sold under the tradename NEO HELIOPAN OS® by the company SYMRISE and of octocrylene sold under the tradename NEO HELIOPAN 303® by the company SYMRISE; and
a combination thereof.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

The composition of the invention should be cosmetically or dermatologically acceptable, i.e., it should contain a non-toxic physiologically acceptable medium and should be able to be applied to the eyelashes of human beings.

According to preferred embodiments of the present invention, methods of caring for, and/or making up, keratinous material by applying compositions of the present invention to the keratinous material in an amount sufficient to care for, and/or to make up, the keratinous material are provided.

Preferably, “making up” the keratin material includes applying at least one coloring agent to the keratin material in an amount sufficient to provide color to the keratin material.

In accordance with the preceding preferred embodiments, the compositions of the present invention are applied topically to the desired keratinous material. The compositions may be applied to the desired area as needed, preferably once or twice daily, more preferably once daily and then preferably allowed to dry before subjecting to contact such as with clothing or other objects (for example, a color coat composition or a topcoat applied over the composition). Preferably, the composition is allowed to dry for about 1 minute or less, more preferably for about 45 seconds or less.

According to preferred embodiments of the present invention, kits comprising, as separate compositions in one or more containers within the kits, (A) a composition, in particular a lip composition, comprising (1) at least one glycerolated silicone resin and (2) a hydrogel-in-oil dispersion; and (3) preferably, at least one colorant; and (B) at least one topcoat or primer coat composition are provided. Preferably, the composition (A) further comprises at least one active agent.

Compositions (A) and (B) may be contained in different portions or sections of the same container within the kit. However, compositions (A) and (B) may also be in different containers with the kit.

According to preferred embodiments of the present invention, sets, as applied onto keratinous material, comprising (A) at least one first layer of at least one primer coat and/or topcoat; and (B) at least one second layer of at least one composition, in particular a lip composition, comprising (1) at least one glycerolated silicone resin and (2) a hydrogel-in-oil dispersion; and (3) preferably, at least one colorant are provided. Preferably, the at least one second layer is applied over the at least one first layer (in which case the first layer is a primer coat composition). However, if the at least one second layer is applied under the at least one first layer, the at least one first layer is a topcoat layer. Preferably, the layer (B) further comprises at least one active agent.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Examples

### Example I—Sample Formulations

Cosmetic lip formulations were manufactured, using the compositions shown below.
Control 1A had 20% glycerolated MQ resin content, but no hydrogel-in-oil dispersion.
Control 1B had 30% glycerolated MQ resin content, but no hydrogel-in-oil dispersion.
Control 2 had hydrogel-in-oil dispersion, but no glycerolated MQ resin.
Inventive compositions A-I contained the following amounts of hydrogel-in-oil dispersion and glycerolated MQ resin, respectively:
Inventive A—12% active to 12% active
Inventive B—12% active to 10% active
Inventive C—12% active to 5% active
Inventive D—10% active to 12% active
Inventive E—10% active to 10% active
Inventive F—10% active to 5% active
Inventive G—5% active to 12% active
Inventive H—5% active to 10% active
Inventive I—5% active to 5% active

The compositions also contained pigments, volatile hydrocarbon oil (isododecane) and thickener. The complete formulations were:

| INCI US | control 1A | control 1B | control 2 | Inventive A 12:12 |
|---|---|---|---|---|
| hydrogel-in- | 0.000 | 0.00 | 58.82 | 35.29 |
| oil dispersion | | | | |
| (33% active) | | | | |
| Glycerolated | 40.000 | 60.000 | 0.000 | 24.000 |
| MQ resin in | | | | |
| isododecane | | | | |
| (50% active) | | | | |
| Pigment | 6.667 | 6.667 | 6.667 | 6.667 |
| grind in | | | | |
| isododecane | | | | |
| (30% active) | | | | |
| Thickener in | 33.330 | 33.330 | 33.330 | 33.330 |
| isododecane | | | | |
| (9% active) | | | | |
| Isododecane | QS | QS | QS | QS |
| | 100.00 | 100.00 | 100.00 | 100.00 |

| | Inventive B | Inventive C | Inventive D | Inventive E |
|---|---|---|---|---|
| INCI US | 12:10 | 12:5 | 10:12 | 10:10 |
| hydrogel-in- | 35.290 | 35.290 | 29.41 | 29.41 |
| oil dispersion | | | | |
| (33% active) | | | | |
| Glycerolated | 20.000 | 10.000 | 24.000 | 20.000 |
| MQ resin in | | | | |
| isododecane | | | | |
| (50% active) | | | | |
| Pigment | 6.667 | 6.667 | 6.667 | 6.667 |
| grind in | | | | |
| isododecane | | | | |
| (30% active | | | | |
| Thickener in | QS | QS | QS | QS |
| isododecane | | | | |
| (9% active) | | | | |
| Isododecane | 4.713 | 14.713 | 6.593 | 10.593 |
| | 100.00 | 100.00 | 100.00 | 100.00 |

| | Inventive F | Inventive G | Inventive H | Inventive I |
|---|---|---|---|---|
| INCI US | 10:5 | 5:12 | 5:10 | 5:5 |
| hydrogel-in- | 29.41 | 14.71 | 14.71 | 14.71 |
| oil dispersion | | | | |
| (33% active) | | | | |
| Glycerolated | 10.000 | 24.000 | 20.000 | 10.000 |
| MQ resin in | | | | |
| isododecane | | | | |
| (50% active) | | | | |
| Pigment | 6.667 | 6.667 | 6.667 | 6.667 |
| grind in | | | | |
| isododecane | | | | |
| (30% active | | | | |
| Thickener in | 33.330 | 33.330 | 33.330 | 33.330 |
| isododecane | | | | |
| (9% active) | | | | |
| Isododecane | QS | QS | QS | QS |
| | 100.00 | 100.00 | 100.00 | 100.00 |

### Example 2—Testing Methods

### Swellability (Caliper Readings)

A 3 mL drawdown was cast on abrasion paper and dried on bench at ambient temperature to yield a film which was measured for thickness using a caliper. Approximately 0.25 g water was added and allowed to evaporate from the top of the film for approximately 1.5 hours, and the film was measured again, with the difference in the two measurements being the caliper-based “swelling” measurements shown in the Table below.

### Film Disruption

Each formulation was applied to several Black Scrub Panels P121-10N #5015 byko-chart using a 1 mil drawdown to produce a film. The films were allowed to dry overnight at ambient temperature. To evaluate the film samples, 6 drops of fluids (two drops of olive oil, two drops of artificial saliva, and two drops of 2% acetic acid) were placed on different sections of the film, and then allowed to stand for 10 minutes. The amount of material removed by the drops themselves was evaluated. In addition, a cotton round was used 15 times to gently wipe off each type of drop. It was then observed how much product was wiped off and whether/if the product moved on the byko-chart substrate. The ratings for these determinations were based on a scale of 0 (no removal) to 3 (substantial removal). Specifically, 1=no removal, 1.5=slight removal, 2-half removal 2.5=significant removal, 3=full removal. Various film disruption fluids (olive oil, artificial saliva, acetic acid) were used to determine wear properties, but they all encompassed the same techniques. The experiment was done in duplicate, with results being reported as the average of two readings.

Results from the testing are reported below.

| | | | | | | | Ave | | |
|---|---|---|---|---|---|---|---|---|---|
| | Oil | | | Saliva | Acetic | A.A. | Wear | Caliper | Caliper |
| | Oil | Cotton | Saliva | Cotton | Acid | Wipe | Score | Swelling | Removal |
| Control 1A | 1.00 | 1.75 | 1.00 | 1.13 | 1.00 | 1.50 | 1.23 | NA | 1.00 |
| Control 1B | 1.00 | 1.50 | 1.00 | 1.00 | 1.00 | 1.25 | 1.13 | NA | 1.00 |
| Control 2 | 1.00 | 3.00 | 2.50 | 2.38 | 1.75 | 2.00 | 2.10 | 0.052 | 1.00 |
| Inventive A | 1.00 | 1.50 | 1.88 | 1.50 | 1.13 | 1.50 | 1.42 | 0.014 | 4.00 |
| Inventive B | 1.00 | 1.50 | 1.75 | 1.50 | 1.25 | 1.50 | 1.42 | 0.023 | 3.00 |
| Inventive C | 1.00 | 3.00 | 1.63 | 1.63 | 1.25 | 2.00 | 1.75 | 0.014 | 5.00 |
| Inventive D | 1.00 | 1.38 | 1.13 | 1.13 | 1.13 | 1.50 | 1.21 | 0.023 | 2.00 |
| Inventive E | 1.00 | 1.63 | 1.50 | 1.50 | 1.25 | 1.50 | 1.40 | 0.016 | 2.00 |
| Inventive F | 1.00 | 3.00 | 1.75 | 1.63 | 1.25 | 2.00 | 1.77 | 0.016 | 3.00 |
| Inventive G | 1.00 | 2.00 | 1.00 | 1.38 | 1.00 | 1.63 | 1.33 | −0.002 | 1.00 |
| Inventive H | 1.00 | 2.38 | 1.00 | 1.50 | 1.13 | 1.75 | 1.46 | 0.002 | 1.00 |
| Inventive I | 1.00 | 3.00 | 1.00 | 1.88 | 1.25 | 2.25 | 1.73 | −0.003 | 2.00 |

Some general observations concerning thee above results are as follows:
Comparing Inventives A, B, D, and E having higher film former concentration vs Inventives C, F, H, and I with lower concentrations, the average wear scores were 1.42, 1.42, 1.21, 1.42 vs 1.75, 1.77, 1.46, 1.73 respectively, where the closer the number is to 1, the better the wear performance;
As glycerolated MQ resin content decreased with consistent concentration of hydrogel-in-oil dispersion, an increase in transfer to the cotton round occurred for oil, acetic acid and artificial saliva testing;
The hydrogel-in-oil dispersion control (control 2) was significantly affected by artificial saliva testing, with intense transfer. Similarly, high transfer to the cotton round occurred.
Among the inventive compositions, better performers in terms of average wear transfer results were, compositions having concentrations of hydrogel-in-oil dispersion to glycerolated MQ resin hydrogel-in-oil dispersion to glycerolated MQ resin of 12:12; 12:10; 10:12; 10:10 and 5:12, with the 5:12 inventive performing slightly less well with respect to swellability;
Inventives with the lowest concentration of glycerolated MQ resin showed relatively more transfer to the cotton rounds for oil, acetic acid and saliva testing (See, Inventives C, F and I); and
Inventives having the lowest concentration of hydrogel-in-oil dispersion but the middle or highest concentration of glycerolated MQ resin showed relatively less transfer resistance, particularly in saliva testing.

Relatively speaking, preferred performance resulted from Inventives B, D, A and E with the best balance of wear and swellability and removal properties.

What is claimed is:

1. A cosmetic composition for use on lips, comprising:
A. a dispersion comprising:
(i) an ethylenic polymeric core particle obtained by polymerization of monomers of:
(a) (C₁-C₄) alkyl (C₁-C₄) (alkyl)acrylate in which the (C₁-C₄) alkyl group(s) are from optionally substituted by one or more hydroxy groups and/or (di)(C₁-C₄)(alkyl) amino groups;
(b) poly [oxy (C₁-C₄) alkylene] (C₁-C₄) (alkyl)acrylate; and/or
(c) ethylenic monomers comprising one or more groups selected carboxy, anhydride, phosphoric acid, and sulfonic acid;
(ii) a polymeric stabilizing agent chosen from:
(d) ethylenic polymers of (C₁-C₆) (alkyl) acrylate of (C₃-C₁₂) cycloalkyl monomers; and/or
(e) copolymers of ethylenic monomers of (C₁-C₆) (C₃-C₁₂) cycloalkyl (alkyl)acrylate and (C₁-C₄) alkyl (C₁-C₄) (alkyl)acrylate; and
(iii) a fatty substance that is liquid at 20° C. and 1 atmosphere;
B. a film forming component comprising at least one glycerolated silicone resin.

2. The cosmetic composition according to claim 1, wherein the film forming component comprises at least one glycerolated silicone resin.

3. The cosmetic composition according to claim 1, wherein the film forming component comprises at least one glycerolated MQ resin.

4. The cosmetic composition according to claim 1, wherein the film forming component comprises at least one additional film former in addition to the at least one glycerolated silicone resin.

5. The cosmetic composition according to claim 1, wherein the dispersion further comprises an additional particle, the additional particle having a different chemical structure from the ethylenic polymeric core particle, the additional particle obtained by polymerization of monomers chosen from ethylenic monomers of (a), (b), and/or (c).

6. The cosmetic composition according to claim 1, wherein the ethylenic polymers of (C₁-C₆) (alkyl) acrylate of (C₃-C₁₂) cycloalkyl monomers are ethylenic homopolymers of (C₁-C₆) (alkyl) acrylate of (C₃-C₁₂) cycloalkyl.

7. The cosmetic composition according to claim 3, wherein the ethylenic polymers of (C₁-C₆) (alkyl) acrylate of (C₃-C₁₂) cycloalkyl monomers are ethylenic homopolymers of (C₃-C₁₂) cycloalkyl (meth) acrylate.

8. The cosmetic composition according to claim 1, wherein the copolymers of ethylenic monomers of (C₁-C₆) (C₃-C₁₂) cycloalkyl (alkyl)acrylate and (C₁-C₄) alkyl (C₁-C₄) (alkyl)acrylate are copolymers of (C₃-C₁₂) cycloalkyl (meth) acrylate and (C1-C4) alkyl (meth) acrylate.

9. The cosmetic composition according to claim 1, wherein the fatty substance is a volatile hydrocarbon, preferably isododecane.

10. The cosmetic composition according to claim 1, further comprising at least one hydrophilic active agent or at least one colorant.
